(19) United States
(12) Patent Application Publication
(10) Pub. No.: US 2004/0102511 A1
(43) Pub. Date: May 27, 2004

(54) Substituted pyrrole derivatives

(76) Inventors: Jitendra Sattigeri, Gurgaon (IN); Mohammad Salman, Gurgaon (IN); Sachin Sethi, Yamuna Nagar (IN); Shobhanna Rawat, Faridabad (IN); Yatendra Kumar, Gurgaon (IN)

Correspondence Address: Jayadeep R. Deshmukh, Esq., Ranbaxy Pharmaceuticals Inc., Suite 2100, 600 College Road East, Princeton, NJ, 08540, US

(21) Appl. No.: 10/429,552

(22) Filed: May 5, 2003

(30) Foreign Application Priority Data
Nov. 21, 2002 (IN) 1176/DEL/2002

(51) Int. Cl. A61K 31/4025; A61K 31/401; C 07D 4 5/02

(52) U.S. Cl. 514/422; 514/423; 548/530; 548/517

(57) ABSTRACT

The present invention relates to substituted pyrrole derivatives, which can be used as 3-hydroxy-3-methylglutaryl-coenzyme A (HMG-CoA) reductase inhibitors. Compounds disclosed herein can function as cholesterol lowering agents and can be used for the treatment of cholesterol-related diseases and related symptoms. Processes for the preparation of disclosed compounds are provided, as well as, pharmaceutical compositions containing the disclosed compounds, and methods of treating cholesterol-related diseases and related symptoms.

### FIELD OF THE INVENTION

The present invention relates to substituted pyrrole derivatives, which can be used as 3-hydroxy-3-methylglutaryl-coenzyme A (HMG-CoA) reductase inhibitors.

Compounds disclosed herein can function as cholesterol lowering agents and can be used for the treatment of cholesterol-related diseases and related symptoms. Processes for the preparation of disclosed compounds are provided, as well as, pharmaceutical compositions containing the disclosed compounds, and methods of treating cholesterol-related diseases and related symptoms.

### BACKGROUND OF THE INVENTION

Cardiovascular disease and its associated maladies, dysfunctions and complications are a principal cause of disability and the chief cause of death. One specific aspect of cardiovascular disease, significantly contributing to these pathophysiologic conditions, is atherosclerosis, which has been generally recognized as the leading health care problem both with respect to mortality and health care costs.

Atherosclerosis is characterized by the deposition of fatty substances, primarily cholesterol, resulting in plaque formation on the inner surface of the arterial wall and degenerative change to the arteries.

It is now well established that vascular blockage and cardiovascular disorders including myocardial infarction, coronary heart disease, hypertension and hypotension, cerebrovacular disorders including stroke, cerebral thrombosis and memory loss due to stroke; peripheral vascular disease and intestinal infarction are caused by blockage of arteries and arterioles by atherosclerosis plaque. Atherosclerotic plaque formation is multi-factorial in its production. Hypercholesterolemia, especially elevated level of low-density lipoprotein (LDL) cholesterol (LDL) is an important risk factor for atherosclerosis and arteriosclerosis and associated diseases.

The HMG-CoA reductase inhibitors (statins) have been used in reducing blood levels of LDL cholesterol. Cholesterol is produced via the mevalonic acid pathway. Reducing the formation of mevalonic acid, a precursor to cholesterol, leads to a corresponding decrease in hepatic cholesterol biosynthesis with a reduction in the cellular pool of cholesterol.

U.S. Pat. No. 4,681,893 assigned to Warner-Lambert, discloses certain trans-6-[2-(3-,or 4-carboxamido-substituted pyrrole-1-yl)alkyl]-4-hydroxypyran-2-ones and the corresponding ring-opened hydroxy acids derived therefrom, including trans(±)-5-(4-fluorophenyl)-2-(1-methylethyl)-N,4-diphenyl-1-[2-tetrahydro-4-hydroxy-6-oxo-2H-pyran-2-yl)ethyl]-1H-pyrrole-3-carboxamide, which are inhibitors of 3-hydroxy-3-methylglutaryl-coenzyme A reductase (HMG-CoA), an important coenzyme catalyzing the intracellular synthesis of cholesterol.

The U.S. Pat. No. 5,273,995 assigned to Warner Lambert, relates to the optically pure (R, R) form of the ring-opened acid of trans-5-(4-fluorophenyl)-2-(1-methylethyl-N,4-diphenyl-1-[2-tetrahydro-4-hydroxy-6-oxo-2H-pyran-2-yl)ethyl]-1 H-pyrrole-3-carboxamide that is [R-(R*, R*)]-2-(4-fluorophenyl)-β,δ-dihydroxy-5-(1-methylethyl)-3-phenyl-4-[(phenylamino)carbonyl]-1H-pyrrole-1-heptanoic acid, pharmaceutically acceptable salts thereof, specifically its calcium salt (Atorvastatin, Lipitor®), which is currently being used for the treatment of hypercholesterolemia.

### SUMMARY OF THE INVENTION

However, the compounds disclosed herein, have inhibitory activity superior to the calcium salt of [R-(R*, R*)]-2-(4-fluorophenyl)-β,δ-dihydroxy-5-(1-methylethyl)-3-phenyl-4-[(phenylamino)carbonyl]-1H-pyrrole-1-heptanoic acid (atorvastatin). These compounds exhibited potency greater than atorvastatin and are about 2-to 10-fold more potent than atorvastatin in inhibiting HMG-CoA reductase, the key rate limiting steps in the biosynthetic pathway. Therefore, these compounds hold promise for the treatment of hypercholesterolemia and hyperlipidemia.

Accordingly, substituted pyrrole derivatives, which can be used for the treatment of cholesterol-related diseases or related symptoms thereof, and process for the synthesis of these compounds as provided.

Pharmaceutically acceptable salts, pharmaceutically acceptable solvates, polymorphs or N-oxides of these compounds having the same type of activity are alos provided.

Pharmaceutical compositions containing the compounds, and which may also contain pharmaceutically acceptable carriers or diluents, which can be used for the treatment of cholesterol-related disease or related symptoms thereof.

Other aspects will be set forth in the accompanying description which follows and in the part will be apparent from the description or may be learnt by the practice of the invention.

In accordance with another aspect, there is provided a method for treating a mammal suffering from cholesterol-related disease, diabetes and related disease, cerebrovascular disease or cardiovascular disease, comprising administering to a mammal a therapeutically effective amount of compounds disclosed herein.

The compounds of the present invention can be used for treating arteriosclerosis, atherosclerosis, hypercholesterolemia, hyperlipidemia, hyperlipoproteinemia, hypertriglyceridemia, hypertension, stroke, ischemia, endothelium dysfunction, peripheral vascular disease, peripheral arterial disease, coronary heart disease, myocardial infarction, cerebral infarction, myocardial microvascular disease, dementia, Alzheimer's disease, osteoporosis and/or osteopenia, angina, or resterosis.

In accordance with yet another aspect, there are provided processes for the preparation of the compounds described herein.

### DETAILED DESCRIPTIN OF THE INVENTION

Substituted pyrrole derivatives of Formula I,

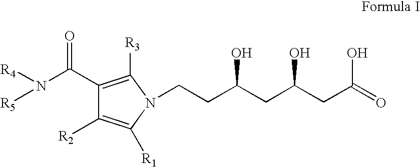

its lactone form, pharmaceutically acceptable salts, pharmaceutically acceptable solvates, polymorphs or N-oxide wherein

R₁ is 4-fluorophenyl, 2,4-difluorophenyl, 3,4-difluorophenyl or cyclohexyl;

R₂ is phenyl, 4-fluorophenyl, 2,4-difluorophenyl, 4-methylphenyl or 4-trifluoromethylphenyl;

R₃ is isopropyl or cyclopropyl;

R₄ is hydrogen or methyl;

R₅ is phenyl, 2- fluorophenyl 3- fluorophenyl, 4-fluorophenyl, 2-pyridyl, 3-pyridyl, 4-pyridyl, 2-cyanophenyl, 4-cyanophenyl, 2,4-difluorophenyl, or 4-trifluoromethylphenyl with the provisio that simultaneously R₁, R₂, R₃, R₄ and R₅ can not be respectively, 4-fluorophenyl, phenyl, isopropyl, hydrogen and phenyl.

An illustrative list of compounds of the invention are listed below (also shown in Table I):

(3R,5R)-7-[2-(4-Fluorophenyl)-5-isopropyl-3-phenyl-4-[(N-methyl-N-phenylamino)carbonyl]-pyrrol-1-yl]-3,5-dihydroxy-1-heptanoic acid calcium salt (Compound No. 1),

(3R,5R)-7-[2-(4-Fluorophenyl)-5-isopropyl-3-phenyl-4-[(2-fluorophen ylamino)carbonyl]-pyrrol-1-yl]-3,5-dihydroxy-1-heptanoic acid calcium salt (Compound No. 2),

(3R,5R)-7-[2-(4-Fluorophenyl)-5-isopropyl-3-phenyl-4-[(3-fluorophen ylamino)carbonyl]-pyrrol-1-yl]-3,5-dihydroxy 1-heptanoic acid calcium salt(Compound No. 3),

(3R,5R)-7-[2-(4-Fluorophenyl)-5-isopropyl-3-phenyl-4-[(4-fluorophenylamino)carbonyl]-pyrrol-1-yl]-3,5-dihydroxy-1-heptanoic acid calcium salt(Compound No. 4),

(3R,5R)-7-[2-(4-Fluorophenyl)-5-isopropyl-3-phenyl-4-[(pyridin-2-yl-amino)carbonyl]pyrrol-1-yl]-3,5-dihydroxy-1-heptanoic acid calcium salt (Compound No. 5),

(3R,5R)-7-[2-(4-Fluorophenyl)-5-isopropyl-3-phenyl-4-[(pyridin-3-yl-amino)carbonyl]pyrrol-1-yl]-3,5-dihydroxy-1-heptanoic acid calcium salt (Compound No. 6),

(3R,5R)-7-[2-(4-Fluorophenyl)-5-isopropyl-3 -phenyl-4-[(pyridin-4-yl-amino)carbonyl]pyrrol-1-yl]-3,5-dihydroxy-1-heptanoic acid calcium salt(Compound No. 7),

(3R,5R)-7-[2-(4-Fluorophenyl)-5-isopropyl-3-phenyl-4-[(2-cyanophen ylamino)carbonyl]-pyrrol-1-yl]-3,5-dihydroxy-1-heptanoic acid calcium salt (Compound No. 8),

(3R,5R)-7-[2-(4-Fluorophenyl)-5-isopropyl-3-phenyl-4-[(4-cyanophen ylamino)carbonyl]-pyrrol-1-yl]-3,5-dihydroxy-1-heptanoic acid calcium salt (Compound No. 9),

(3R,5R)-7-[2-(4-Fluorophenyl)-5-isopropyl-3-phenyl-4-[(2,4-difluorophenylamino)carbonyl]-pyrrol-1-yl]-3,5-dihydroxy-1-heptanoic acid calcium salt (Compound No. 10),

(3R,5R)-7-[2-(4-Fluorophenyl)-5-isopropyl-3 -phenyl-4-[(4-trifluoromethylphenylamino)carbonyl]-pyrrol-1-yl]-3,5-dihydroxy-1-heptanoic acid calcium salt (Compound No. 11),

(3R,5R)-7-[2-(2,4-Difluorophenyl)-5 -isopropyl-3-phenyl-4-[(phenylamino)carbonyl]-pyrrol-1-yl]-3,5-dihydroxy-1-heptanoic acid calcium salt (Compound No. 12),

(3R,5R)-7-[2-(3,4-Difluorophenyl)-5 -isopropyl-3-phenyl-4-[(phenylamino)carbonyl]-pyrrol-1-yl]-3,5-dihydroxy-1-heptanoic acid calcium salt (Compound No. 13),

(3R,5R)-7-[2-Cyclohexyl-5-isopropyl-3-phenyl-4-[(phenylamino)carbonyl]-pyrrol-1-yl]-3,5-dihydroxy-1-heptanoic acid calcium salt (Compound No. 14),

(3R,5R)-7-[2-(4-Fluorophenyl)-5-isopropyl-3-(2,4-difluorophenyl)-4-[(phenylamino)carbonyl]-1H-pyrrol-3,5-dihydroxy-1-heptanoic acid calcium salt(Compound No. 15),

(3R,5R)-7-[2,3-Di-(4-fluorophenyl)-5-isopropyl-4-[(phenylamino) carbonyl]-pyrrol-1-yl]-3,5-dihydroxy-1-heptanoic acid calcium salt (Compound No. 16),

(3R,5R)-7-[2-(4-Fluorophenyl)-5-isopropyl-3 -(4-methylphenyl)-4-[(phenylamino)carbonyl]-pyrrol-1-yl]-3,5-dihydroxy-1-heptanoic acid calcium salt (Compound No. 17),

(3R,5R)-7-[2-(4-Fluorophenyl)-5-isopropyl-3-(4-trifluoromethylphenyl)-4-[(phenylamino)carbonyl]-pyrrol-1-yl]-3,5-dihydroxy-1-heptanoic acid calcium salt (Compound No. 18),

(3R,5R)-7-[2-(4-Fluorophenyl)-5-cyclopropyl-3-phenyl-4-[(phenylamino)carbonyl]-pyrrol-1-yl]-3,5-dihydroxy-1-heptanoic acid calcium salt (Compound No. 19),

TABLE I

| | |
|---|---|
| 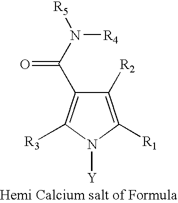 | 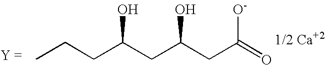 |

| Compound | | | | | |
|---|---|---|---|---|---|
| No. | R₁ | R₂ | R₃ | R₄ | R₅ |
| 1 | 4-Fluorophenyl | Phenyl | Isopropyl | Methyl | Phenyl |
| 2 | 4-Fluorophenyl | Phenyl | Isopropyl | Hydrogen | 2-Fluorophenyl |
| 3 | 4-Fluorophenyl | Phenyl | Isopropyl | Hydrogen | 3-Fluorophenyl |
| 4 | 4-Fluorophenyl | Phenyl | Isopropyl | Hydrogen | 4-Fluorophenyl |
| 5 | 4-Fluorophenyl | Phenyl | isopropyl | Hydrogen | 2-Pyridyl |
| 6 | 4-Fluorophenyl | Phenyl | Isopropyl | Hydrogen | 3-Pyridyl |
| 7 | 4-Fluorophenyl | Phenyl | Isopropyl | Hydrogen | 4-Pyridyl |
| 8 | 4-Fluorophenyl | Phenyl | Isopropyl | Hydrogen | 2-Cyanophenyl |
| 9 | 4-Fluorophenyl | Phenyl | Isopropyl | Hydrogen | 4-Cyanophenyl |
| 10 | 4-Fluorophenyl | Phenyl | Isopropyl | Hydrogen | 2,4-Difluorophenyl |
| 11 | 4-Fluorophenyl | Phenyl | Isopropyl | Hydrogen | 4-Trifluoromethylphenyl |
| 12 | 2,4-Difluorophenyl | Phenyl | Isopropyl | Hydrogen | Phenyl |
| 13 | 3,4-Difluorophenyl | Phenyl | Isopropyl | Hydrogen | Phenyl |
| 14 | Cyclohexyl | Phenyl | Isopropyl | Hydrogen | Phenyl |
| 15 | 4-Fluorophenyl | 2,4-Difluorophenyl | Isopropyl | Hydrogen | Phenyl |
| 16 | 4-Fluorophenyl | 4-Fluorophenyl | Isopropyl | Hydrogen | Phenyl |
| 17 | 4-Fluorophenyl | 4-Methylphenyl | Isopropyl | Hydrogen | Phenyl |
| 18 | 4-Fluorophenyl | 4-Trifluoromethylphenyl | Isopropyl | Hydrogen | Phenyl |
| 19 | 4-Fluorophenyl | Phenyl | Cyclopropyl | Hydrogen | Phenyl |

The compounds described herein may be prepared by techniques well known in the art and familiar to the average synthetic organic chemist. In addition, the compounds of the present invention may be prepared by the following reaction sequences as depicted in Schemes I and II.

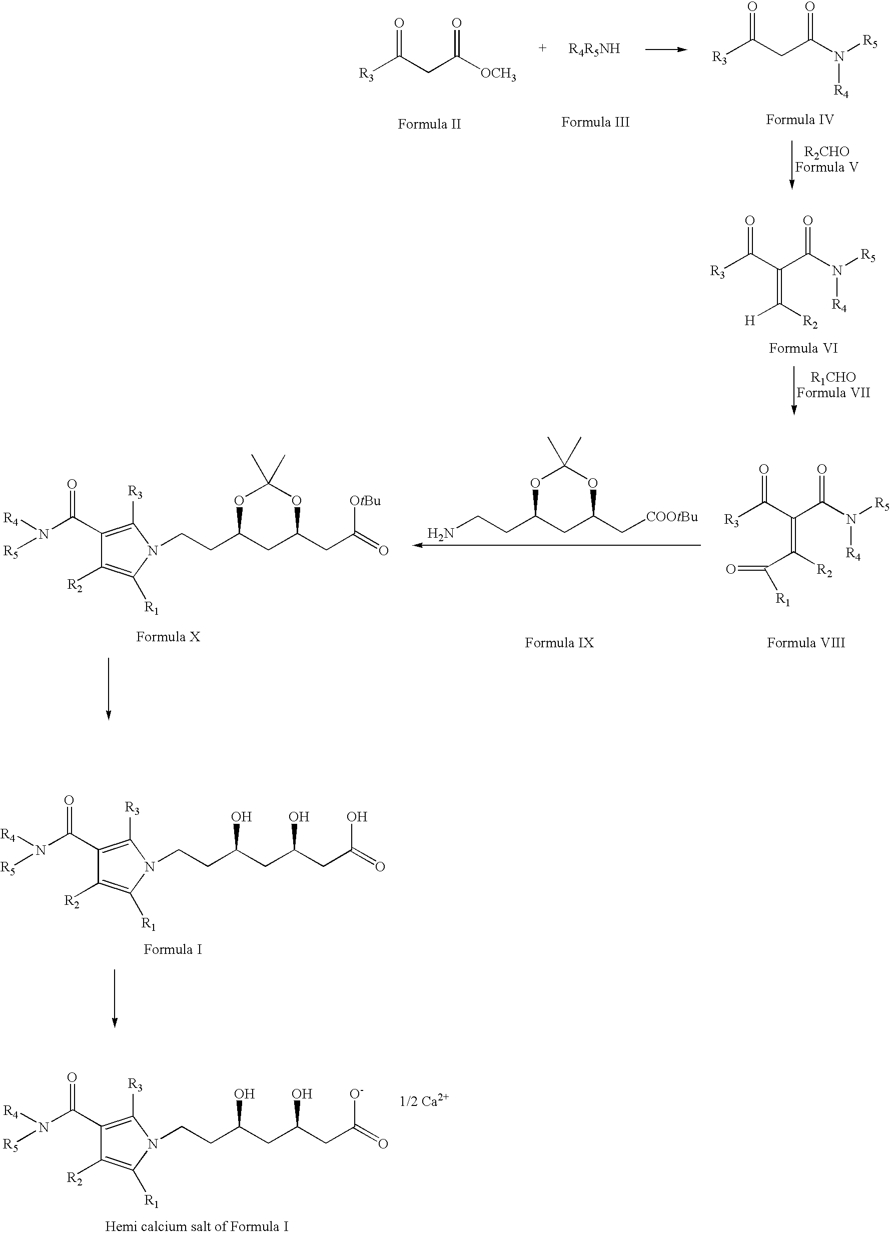

The compounds of Formula I can be prepared according to Scheme I. Thus, a compound of Formula II is reacted with a compound of Formula III wherein R₃, R₄ and R₅ are as defined earlier to give a compound of Formula IV which on reaction with a compound of Formula V (wherein R₂ is as defined earlier) gives a compound of Formula VI, which on treatment with a compound of Formula VII (wherein R₁ is as defined earlier) yields a compound of Formula VIII, which on further reaction with a compound of Formula IX gives a compound of Formula X, which on hydrolysis gives a compound of Formula I, which is then further converted to hemicalcium salt.

The reaction of a compound of Formula II with a compound of Formula III to give a compound of Formula IV is carried out in a suitable solvent such as xylene or toluene.

The reaction of a compound of Formula II with a compound of Formula III is carried out in the presence of a suitable base such as diethylamine, triethylamine, pyridine or 1,2-ethylenediamine.

The reaction of a compound of Formula IV with an aldehyde of Formula V to give a compound of Formula VI is carried out in a suitable solvent such as hexane, heptane, dichloromethane or toluene.

The reaction of a compound of Formula IV with an aldehyde of Formula V is carried out in the presence of an organic base such as piperidine, pyridine and β-alanine or an organic acid such as glacial acetic acid and benzoic acid.

The reaction of a compound of Formula VI with an aldehyde of Formula VII to give a compound of Formula VIII is carried out in the presence of a suitable catalyst such as sodium cyanide, thiazolium bromide or thiazolium chloride in a suitable solvent such as methanol, ethanol, propanol or isopropanol.

The reaction of a compound of Formula VI with an aldehyde of Formula VII is carried out in the presence of a suitable base such as diethylamine, triethylamine or pyridine.

The reaction of a compound of Formula VIII with a compound of Formula IX to give a compound of Formula X is carried out in a suitable solvent selected from the group comprising of xylene and toluene.

The reaction of a compound of Formula VIII with a compound of Formula IX is carried out in the presence of an organic acid such as pivalic acid or p-toluene sulfonic acid.

The conversion of a compound of Formula X to a compound of Formula I is carried out in a two step manner involving an initial acid-catalysed cleavage of ketal followed by base-catalysed hydrolysis of tert-butyl ester. The acid can be a mineral acid such as aqueous hydrochloric acid and the base can be lithium hydroxide, sodium hydroxide or potassium hydroxide.

The compound of Formula I can be converted into its corresponding calcium salt (hemi calcium salt of Formula I) by following procedures well-known to a person of ordinary skill in the art.

The calcium salts of compound of Formula I can also be prepared from the corresponding lactones form of Formula I by following the procedures well-known in the art.

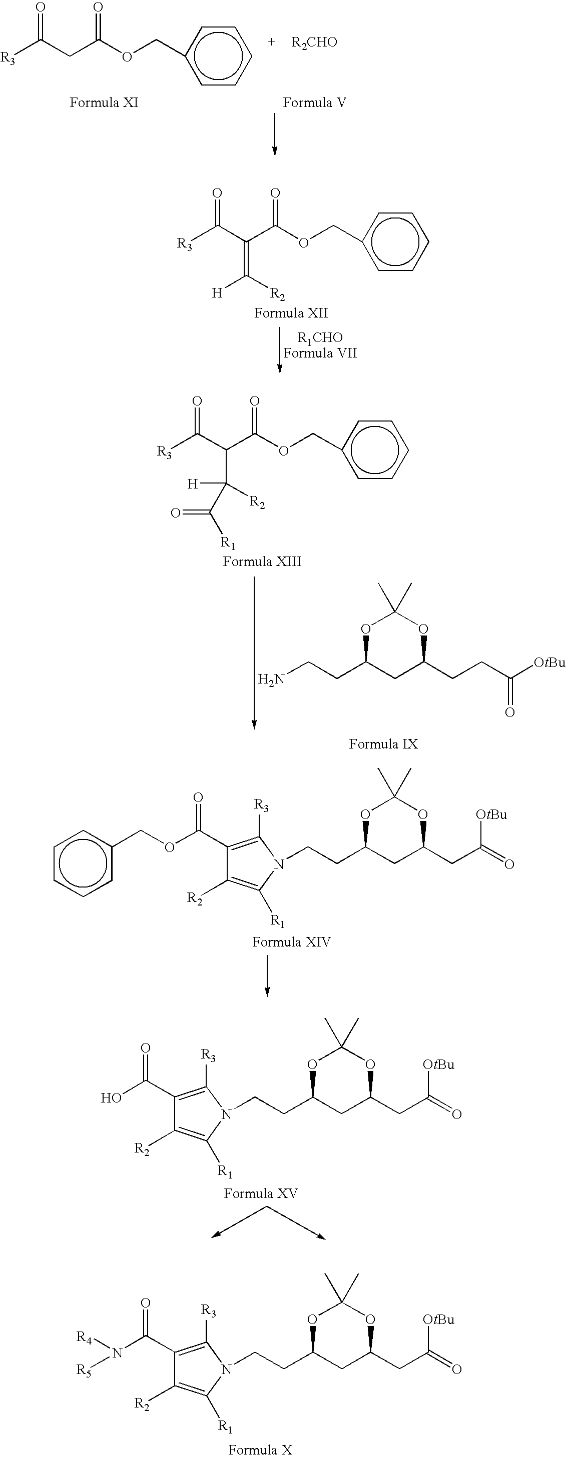

In Scheme II the compound of Formula I can be prepared by reacting a compound of Formula XI with a compound of Formula V to give a compound of Formula XII wherein R₂ and R₃ are as defined earlier, the compound of Formula XII on reaction with a compound of Formula VII (wherein R₁ is as defined earlier) gives a compound of Formula XIII, which on treatment with a compound of Formula IX yields a compound of Formula XIV, which on debenzylation gives a compound of Formula XV, which on conversion to corresponding acid chloride (Path a) or reacting with alkyl chloroformate (Path b) followed by reaction with a compound of Formula III gives a compound of Formula X, which on hydrolysis gives a compound of Formula I, which can then be further converted to the hemicalcium salt of Formula I by following procedures well-known in the art.

The reaction of a compound of Formula XI with an aldehyde of Formula V to give a compound of Formula XII is carried out in a suitable solvent such as of xylene, toluene, heptane, hexane or dichloromethane.

The reaction of a compound of Formula XI with a compound of Formula V is carried out in the presence of an organic base such as triethylamine, pyridine or piperidine, β-alanine or an organic acid such as glacial acetic acid or benzoic acid.

The reaction of a compound of Formula XII with an aldehyde of Formula VII to give a compound of Formula XIII is carried out in a suitable solvent such as methanol, ethanol, propanol or isopropanol.

The reaction of a compound of Formula XII with an aldehyde of Formula VII is carried out in the presence of an organic base such as diethylamine, triethylamine, piperidine or pyridine.

The reaction of a compound of Formula XII with an aldehyde of Formula VII to give a compound of Formula XIII is carried out in the presence of a suitable catalyst such as sodium cyanide, thiazolium bromide or thiazolium chloride.

The reaction of a compound of Formula XIII with an amine of Formula IX to give a compound of Formula XIV is carried out in the presence of an acid such as pivalic acid and p-toluene sulfonic acid and a suitable solvent such as hexane, heptane, toluene OR tetrahydrofuran.

The debenzylation of a compound of Formula XIV to give a compound of Formula XV is carried out in the presence of a catalyst such as palladium on carbon and hydrogen in a suitable solvent selected from the group comprising of methanol, ethanol, propanol and dioxane.

The conversion of compound of formula XV to its corresponding acid chloride (Path a) is carried with any suitable chlorinating agent such as oxalyl chloride in presence of suitable solvent such as benzene, toluene and xylene followed by reaction with a compound of Formula III to give a compound of Formula X in the presence of a suitable solvent such as benzene and an organic base such as triethylamine or pyridine.

Reaction of compound of Formula XV with alkyl chloroform ate (Path b) such as ethyl chloroformate, isopropyl chloroformate or isobutyryl chloroformate is carried out in a suitable solvent such as tetrahydrofuran in the presence of a suitable base such as triethylamine followed by reaction with a compound of Formula III to give a compound of Formula X.

The term “pharmaceutically acceptable” means approved by regulatory agency of the federal or a state government or listed in the U.S. pharmacopeia or other generally recognized pharmacopeia for use in animals, and more particularly for use in humans. The term “pharmaceutically acceptable salts” refer to salts prepared from pharmaceutically acceptable monovalent, divalent or trivalent non-toxic metal or organic base. Example of such metal salts include, but are not limited to, lithium, sodium, potassium, calcium, magnesium, zinc, aluminum, iron and the like. Example of such organic base include, but are not limited to, amino acid, ammonia, mono-alkyl ammonium, dialkyl ammonium, trialkyl ammonium and N-methyl glucamine and the like. The free acid form of compounds of the present invention may be regenerated from the salt form, if desired, by contacting the salt with dilute aqueous solution of an acid such as hydrochloric acid. The base addition salts may differ from the free acid forms of the compounds of this invention in such physical characteristics as solubility and melting point, but can be considered suitable for the purposes disclosed herein.

The term “pharmaceutically acceptable solvates” refers to solvates with water (i.e., hydrates) or pharmaceutically acceptable solvents, for example solvates with, ethanol and the like. Such solvates are also encompassed within the scope of this invention. Furthermore, some of the crystalline forms for compounds described herein may exist as polymorphs and as such are included in the scope of the disclose. Pharmaceutical compositions comprising the compounds disclosed herein, their pharmaceutically acceptable salts, pharmaceutically acceptable solvates, or polymorphs, and pharmaceutically acceptable carriers or excipients.

Compositions provided herein include both those containing one compound disclosed herein and or those that contain two or more of such compounds. These may be suitable for oral or parenteral administration. The compositions may be formulated to provide immediate or sustained release of the therapeutic compounds. The compounds described herein can be administered alone but will generally be administered as an admixture with suitable pharmaceutically acceptable carriers. The term “pharmaceutically acceptable carrier” is intended to include non-toxic, inert solid, semi-solid, liquid filter, diluent, encapsulating material or formulation auxiliary of any type.

Solid form preparations for oral administration may include capsules, tablets, pills, powder, granules and suppositories. For solid form preparations, active compound is mixed with at least one inert, pharmaceutically acceptable excipient or carrier such as sodium citrate, dicalcium phosphate and/or a filter, extenders such as starch, lactose, sucrose, glucose, mannitol and silicic acid; binders such as carboxymethyl cellulose, alginates, gelatins, polyvinylpyrroledinone, sucrose, acacia; disintegrating agents such as agar-agar, calcium carbonate, potato starch, aliginic acid, certain silicates and sodium carbonate; absorption accelerators such as quaternary ammonium compounds; wetting agents such as cetyl alcohol, glycerol, mono stearate adsorbents such as Kaolin; Lubricants such as talc, calcium stearate, magnesium stearate, solid polyethyleneglycol, sodium lauryl sulphate and mixture thereof.

In case of capsules, tablets, pills, the dosage form may also comprise buffering agents.

The solid preparation of tablets, capsules, pills, granules can be prepared with coating and shells such as enteric coating and other coatings well known in the pharmaceutical formulating art.

Liquid form preparations for oral administration include pharmaceutically acceptable emulsions, solution, suspensions, syrups and elixirs. For liquid form preparations, active compound is mixed with water or other solvent, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethyl formamide, oils (such as cottonseed, ground corn, germ, live, caster and sesamine oil), glycerol and fatty acid ester of sorbitan and mixture thereof.

Besides inert diluents, the oral composition can also include adjuvant such as wetting agents, emulsifying agents, suspending agents, sweetening agents, flavoring agents and perfuming agents.

Formulations described herein may be formulated so as to provide either quick, sustained, or delayed release of the active compound after administration to the patient by employing procedures well known to the art. The term “patient” as used herein refers to a mammal, which is the object of treatment, observation or experiment.

The pharmaceutical preparation is in unit dosage form, in such form, the preparation is subdivided into unit doses containing appropriate quantities of the active compound.

The amount of a compound of the present invention that will be effective in the treatment of a particular disorder or condition, and can be determined by standard clinical techniques. In addition, in vitro or in vivo assays may optionally be employed to help identify optional dosage ranges.

Examples set forth below demonstrate general synthetic procedures for the preparation of representative compounds. The examples are provided to illustrate particular aspects of the disclosure and do not constrain the scope of the present invention as defined by the claims.

### EXPERIMENTAL DETAILS

### General Procedure

Scheme I

Step 1: Preparation of β ketoamide-1 (Formula IV)

A mixture of β ketoester (1 equiv), amine (1 equiv) 1,2-ethylene diamine (0.01 equiv) in xylene was refluxed with the azeotropic removal of water. After the completion of reaction, solvent was evaporated & the residue purified on column (silica gel; 100-200 mesh). The following intermediates were prepared following above general procedure:

4-Methyl-3-oxo-pentanoic acid (3-fluoropenyl)-amide

¹H NMR(CDCl₃, 300 MHz): δ 1.17 (d, J=6.9Hz, 6H), 2.74 (sep, J=6.9Hz, 1H), 3.61 (s, 2H), 6.81 (t, J=7.7Hz, 1H), 7.13-7.35 (m, 2H), 7.53 (d, J=10.8Hz, 1H), 9.42 (brs, 1H) Yield: 13%

4-Methyl-3-oxo-pentanoic acid (4-fluorophenyl)-amide

¹H NMR(CDCl₃): δ 1.18 (d, J=6Hz, 6H), 2.74 (sep, J=6Hz, 1H), 3.61 (s, 2H), 7.02 (t, J=9Hz, 2H), 7.49-7.57 (m, 2H) Yield: 93%

4-Methyl-3-oxo-pentanoic acid pyridin-3-yl-amide

¹H NMR(CDCl₃, 300 MHz): δ 1.18 (d, J=9OHz, 6H), 2.73-2.8 (m, 1H), 3.68 (s, 2H), 7.3 (brs, 1H), 8.16 (brs, 1H), 8.37 (brs, 1H), 8.68 (brs, 1H), 9.59 (brs, 1H) Yield=35%

4-Methyl-3-oxo-pentanoic acid pyridin-4-yl-amide

¹H NMR(CDCl₃, 300 MHz): δ 1.18 (d, J=6.9Hz, 6H), 2.74 (sep. J=6.9Hz, 1H), 3.6 (s, 2H), 7.52 (d, J=5.7, 2H), 8.5 (d, J=6.0Hz, 2H), 9.74 (brs, 1H) Yield=37%

4-Methyl-3-oxo-pentanoic acid (4-cyanophenyl)-amide

¹H NMR(CDCl₃): δ 1.19 (d, J=6.6Hz, 6H), 2.74 (Sep, J=6.6Hz, 1H), 3.64 (s, 2H), 7.62 (d, J=8.7Hz, 2H), 7.70 (d, J=8.7Hz, 2H), 9.66 (brs, 1H) MS (+ve ion mode): m/z 231 [M+1] Yield: 25%

4-Methyl-3-oxo-pentanoic acid (2,4-difluoropenyl)-amide

¹H NMR(CDCl₃, 300 MHz): δ 1.18 (D, j=6.9Hz, 6H), 2.74 (sept, J=6.9Hz, 1H), 3.64 (s, 2H), 6.82-6.91 (m, 2H), 8.16-8.24 (m, 1H), 9.47 (brs, 1H) Yield=78%

3-Cyclopropyl-3-oxo-N-phenyl-propionamide

¹H NMR(DMSO-d₆, 300 MHz): δ 0.93-0.96 (m, 4H), 2.15-2.19 (m, 1H), 3.66 (s, 2H), 7.06 (t, J=6Hz, 1H), 7.31 (t, J=6Hz, 2H), 7.58 (d, J=6Hz, 2H), 10.13 (s, 1H) MS (+ve ion mode): m/z 204 (M⁺+1) Yield=26%

Step 2: Preparation of β-ketoamide-2 (Formula VI)

β-ketoamide-1 (1 equiv) in hexane was added to β-alanine (0.18 equiv), aldehyde (R₂CHO, 1.1 equiv) and glacial acetic acid (0.16% w/w of β-ketoamide-1). The resulting suspension was heated under reflux with the azeotropic removal of water. The reaction mixture was cooled and product was isolated by filtration. The product was purified by washing the precipitate with hot hexane, water and dried in vacuo to afford β-ketoamide-2.

The following intermediates were prepared following above general procedure

2-Benzylidene-4-methyl-3-oxo-pentanoic acid (3-fluorophenyl)-amide

1H NMR(CDCl₃): δ 1.03 (d, J=6Hz, 6H), 1.22 (d, J=6Hz, 3H), 2.62 (sep, J=6z, 1H), 3.36 (sep, J=6Hz, 0.5H), 6.80-6.90 (m, 1.5H), 7.10-7.16 (m, 0.5H), 7.28-7.72 (m, 12H), 7.74 (brs, 0.5H), 8.18 (s, 1H), 9.18 (brs, 1H) Yield: 75%

2-Benzylidene-4-methyl-3-oxo-pentanoic acid (4-fluoro-phenyl)-amide

¹H NMR(CDCl₃): δ 1.21 (d, J=6Hz, 6H), 3.36 (sep, J=6z, 1H), 7.02 (t, J=6Hz, 2H), 7.30-7.63 (m, 7H), 7.63 (s, 1H), 7.66 (s, 1H) Yield: 38%

2-Benzylidene-4-methyl-3-oxo-pentanoic acid pyridin-3-ylamide

¹H NMR(DMSO-d₆, 300 MHz): δ 1.12 (d, J=6.0Hz, 6H), 7.37-7.43 (m, 5H), 7.43-7.66 (m, 2H), 7.79 (s, 1H), 80.7 (d, J=90Hz, 1H), 8.32 (d, J=3.0Hz, 1H), 8.75 (s, 1H), 10.68 (s, 1H) MS (+ve ion): m/z 295.1 [M⁺+1] Yield=80%

2-Benzylidene-4-methyl-3-oxo-pentanoic acid pyridin-4-ylamide MS (+ve ion): m/z 295.4 [M⁺+1] Yield=91%

2-Benzylidene-4-methyl-3-oxo-pentanoic acid (4-cyanophenyl)-amide

isomer-1: ¹H NMR(CDCl₃): δ 1.03 (d, J=6Hz, 6Hz, 6H), 2.64 (Sep, J=6Hz, 1H), 7.25-7.35 (m, 2H), 7.36-7.48 (m, 3H), 7.65 (d, J=9Hz, 2H), 7.77 (d, J=9Hz, 2H), 3.22 (s, 1H), 9.44 (s, 1H) MS (+ve ion mode): m/z 319 [M+1] Yield: 10%

isomer-2: ¹H NMR(CDCl₃): δ 1.22 (d, J=9Hz, 6H), 3.35 (Sept, J=6Hz, 1H), 7.30-7.75 (m, 10H), 8.23 (s, 1H). MS (+ve ion mode): m/z 319 [M+1] Yield: 22%

2-Benzylidene-4-methyl-3-oxo-pentanoic acid (2,4-difluoropenyl)-amide

¹H NMR(CDCl₃, 300 MHz): (N3: 1 mixture of isomers): δ 1.04 (d, J=6.2H), 1.22 (d, J=6.6Hz, 6H), 2.62 (sept, 3z, 6.9Hz, 0.2), 3.35 (sept, J=6.6Hz, 1H), 6.84-6.91 (m, 2.6H), 7.31-7.73 (m, 6H), 8.18-8.28 (m, 1.3H) MS (+ve ion mode): m/z 330 (M⁺+1) Yield=75%

2-(2,4-Difluoro-benzylidene)-4-methyl-3-oxo-pentanoic acid phenylamide

¹H NMR(CDCl₃): δ 1.23 (d, J=6z, 6H), 3.37 (Sep, J=6Hz, 1H), 6.80-6.90 (m, 2H), 7.16 (t, 6Hz, 1H), 7.35 (t, J=9Hz, 2H), 7.52 (d, J=9Hz, 2H), 7.65-7.72 (m, 2H), 8.00 (brs, 1H) MS (+ve ion mode): m/z 331 [M+1] Yield 39%

2-(4-Fluorobenzylidene)-4-methyl-3-oxo-pentanoic acid phenylamide

¹H NMR(CDCl₃): δ 1.22 (d, J=6.6Hz, 6H), 3.34 (sep, J=6.6Hz, 1H), 7.06 (t, J=8.4Hz, 2H), 7.18 (t, J=7.5Hz, 1H), 7.36 (t, J=7.8Hz, 2H), 7.49-7.65 (m, 6H) MS (+ve ion mode): m/z 312 [M⁺+1] Yield=56%

2-(4-Methylbenzylidene)-4-methyl-3-oxo-pentanoic acid phenylamide

¹H NMR(CDCl₃): δ 1.04 (d, J=9.9Hz, 6H), 1.21 (d, J=6.9Hz, 3H), 2.34 (s, 1.5H), 2.40 (s, 3H), 2.68 (sep. J=6.9Hz, 1H), 3.35 (m, 0.5H), 7.10-7.75 (, 15H), 8.15 (s, 1H), 0.07 (s, 1H) MS (+ve ion mode): m/z 308 [M+1] Yield: 44%

2-(4-Trifluoromethylbenzylidene)-4-methyl-3-oxo-pentanoic acid phenylamide

¹H NMR(CDCl₃): δ 1.23 (d, J=6Hz, 6H), 3.35 (Sep, 5=6Hz, 1H), 7.17 (t, J=9Hz, 1H), 7.36 (t, J=9Hz, 2H), 7.49 (d, J=9Hz, 2H), 7.60-7.78 (m, 6H) MS (+ve ion mode): m/z 362 [M+1] Yield: 58%

2-Cyclopropanecarbonyl-3,N-diphenyl-acryllamide

¹H NMR(CDCl₃, 300 MHz): δ 1.04-1.06 (m, 2H), 1.23 (brs, 2H), 2.46-2.48 (m, 1H), 7.16 (t, J=6Hz, 1H), 7.32-7.37 (m, 5H), 7.52-7.60 (m, 4H), 7.66 (s, 1H), 7.92 (s, 1H). MS (+ve ion mode): m/z 292 (M⁺+1) Yield: 81%

Step 3: Preparation of Diketone (Formula VIII)

β-ketoamide-2 (1 equiv), aldehyde (1.1 equiv), triethylamine (1 equiv) ethanol and thiazolium bromide (0.2 equiv) were placed in a vial. The contents were flushed with N₂ and the vial capped immediately and heated at 78° C. After the completion of reaction, contents were cooled and triturated with ethyl acetate. The organic layer was washed with 6N hydrochloric acid, water, dried over anhydrous sodium sulphate, concentrated on rotavapor and residue purified on column (silica gel, 100-200 mesh)

The following intermediates were prepared following above general procedure:

2-[2-(4-Fluorophenyl)-2-oxo-1-phenyl-ethyl]-4-methyl-3-oxo-pentanoic acid (3-fluorophenyl)amide MS (+ve ion mode): m/z 436 [M⁺+1] Yield: 65%

2-[2-(4-Fluorophenyl)-2-oxo-1-phenyl-ethyl]-4-methyl-3-oxo-pentanoic acid (4-fluorophenyl)-amide

¹H NMR(CDCl₃): δ 1.16 (d, J=6.9Hz, 3H), 1.23 (d, J=6.9Hz, 3H), 2.98 (sep. J=6.9Hz, 1H), 4.53 (d, J=10.8Hz, 1H), 5.35 (d, J=10.8Hz, 1H), 6.92 (t, J=8.7Hz, 2H), 6.98-7.13 (m, 4H), 7.18-7.35 (m, 6H), 7.92-8.05 (m, 2H) Yield: 76%

2-[2-(4-Fluorophenyl)-2-oxo-1-phenyl-ethyl]-4-methyl-3-oxo-pentanoic acid pyridin-3-yl amide

¹H NMR(CDCl₃, 300 MHz): δ 0.69 (d, J=6.0Hz, 0.75H), 0.96 (d, J=9.0Hz, 0.75H), 1.15 (d, J=9.0Hz, 3H), 1.23 (d, J=6.0Hz, 3H), 2.55-2.67 (m, 0.25H), 2.98 (sep, J=6.0Hz, 1H), 4.60 (d, J=9.0Hz, 1H), 4.66 (d, J=9.0Hz, 1H), 5.37 (d, J=9.0Hz, 1H), 5.55 (d, J=9.0Hz, 0.25H), 7.01-7.07 (m, 3.75H), 7.18-7.25 (m, 6.25H), 7.72 (s, 1H), 7.77-7.8 (m, 1H), 7.96-8.0 (m, 2.5H), 8.18 (s, 1H), 8.31 (d, J=3.0Hz, 1H) MS(+ve ion) m/z 419.4 [M⁺+1] Yield: 87%

2-[2-(4-Fluorophenyl)-2-oxo- 1 -phenyl-ethyl]-4-methyl-3-oxo-pentanoic acid pyridin-4-yl amide

¹H NMR(CDCl₃, 300 MHz, isomeric mix 1:3): δ 0.7 (d, J=6.9Hz, 0.99), 0.93 (d, J=6.9Hz, 0.99H), 1.13 (d, J=93Hz, 3H), 1.19 (d, J=9.6Hz, 3H), 2.47-2.65 (m, 033H), 2.99 (sep. J=6.9Hz, 1H), 4.58 (d, J=10.8Hz, 1H), 4.66 (d, J=10.8Hz, 0.33H), 5.35 (d, J=10.5Hz, 1H), 5.51 (d, J=10.5Hz, 0.33H), 7.01-7.07 (m, 3.9H), 7.187.25 (m, 3.6H), 7.42 (d, J=6.0Hz, 0.6H), 7.62 (d, J=6.0Hz, 0.6H), 7.86 (s, 1H), 7.94-7.99 (m, 2.66H), 8.37-8.6 (m, 3.99H) MS (+ve ion ): m/z 419.5 [M⁺+1] Yield: 55%

2-[2-(4-Fluorophenyl)-2-oxo-1-phenyl-ethyl]-4-methyl-3-oxo-pentanoic acid (4-cyanophenyl)-amide

¹H NMR(CDCl₃): δ 1.16 (d, J=6H, 3H), 2.22 (d, J=6Hz, 3H), 2.99 (Sep, J=6Hz, 1H), 4.55 (d, J=12Hz, 1H), 5.33 (d, J=12Hz, 1H), 7.04 (t, J=9Hz, 2H), 7.20-7.42 (m, 6H), 7.43-6.65 (m, 4H), 7.90-8.05 (m, 2H) MS (+ve ion mode): m/z 443 [M+1] Yield: 41%

2-[2-(4-Fluorophenyl)-2-oxo-1-phenylethyl]-4-methyl-3-oxo-pentanoic acid (2,4-difluorophenyl) amide

¹H NMR(CDCl₃, 300 MHz): δ 1.13 (d, J=6.6Hz, 3H), 1.25 (d, J=6.6Hz, 3H), 2.99 (sept, 6.9Hz, 1H), 4.72 (d, J=10.8Hz, 1H), 5.39 (d, J=10.8Hz, 1H), 5.74-6.77 (m, 2H), 7.04 (f, J=8.4Hz, 2H), 7.20-7.32 (m, 3H), 7.65-7.75 (m, 2H), 7.98-7.03 (m, 3H)MS (+ve ion mode): m/z 454 (M⁺+1)-2-[2-(2,4-Difluorophenyl)-2-oxo-1-phenylethyl]-4-methyl-3oxo-pentanoic acid phenylamide ¹H NMR(CDCl₃, 300 MHz): δ 1.12 (d, J=7.2Hz, 3H), 1.24 (d, J=7.2H, 3H), 3.02 (sept, J=6.9Hz, 1H), 4.48 (d, J=10.8Hz, 1H), 5.78 (d, 10.8Hz, 1H), 6.65-6.85 (m, 2H), 6.85-6.95 (m, 1H), 6.95-7.32 (m, 9H), 7.86-7.94 (m, 1H) MS (+ve ion mode): m/z 436 (M⁺+1) Yield: 17%

2-[2-(3,4-Difluorophenyl)-2-oxo-1-phenylethyl]-4-methyl-3-oxo-pentanoic acid phenylamide

¹H NMR(CDCl₃, 300 MHz): δ 1.23 (d, J=6Hz, 3H), 1.15 (d, J=6Hz, 3H), 2.97 (sept, J=6Hz, 1H), 4.50 (d, J=12Hz, 1H), 5.30 (d, J=12Hz, 1H), 7.09-7.22 (m, 6H), 7.25-7.34 (m, 5H), 7.73-781 (m, 2H).MS (+ve ion mode): M/z 436 (M⁺+1) Yield: 49%

2-[2-(4-Fluorophenyl)-2-oxo-1-(4-fluorophenyl)-ethyl]-4-methyl-3-oxo-pentanoic acid phenylamide ¹H NMR(CDCl₃): δ 1.16 (d, J=6.9Hz, 3H), 1.23 (d, J=6.9Hz, 3H), 2.99 (Sep, J=6.6Hz, 1H), 4.49 (d, J=10.5Hz, 1H), 5.36 (d, =10.5Hz, 1H), 6.93-7.19 (m, 7H), 7.21-7.56 (m, 5H), 7.93-7.99 (m, 2H) MS (+ve ion mode): m/z 437 [M⁺+1] Yield: 57%

2-[2-(4-Fluorophenyl)-2-oxo-1-(2,4-difluorophenyl)-ethyl]-4-methyl-3-oxo-pentanoic acid phenylamide ¹H NMR(CDCl₃): δ 1.16 (d, J=9Hz, 3H), 1.21 (d, J=9Hz, 3H), 3.11 (Sep, J=9Hz, 1H), 4.60 (d, J=9Hz, 1H), 5.65 (d, J=12Hz, 1H), 6.65-6.88 (m, 3H), 7.03-7.25 (m, 3H), 7.26 (m, 4H), 7.53 (s, 1H), 7.93-7.98 (m, 2H) MS (+ve ion mode): m/z 454 [M+1] Yield: 52%

2-[2-(4-Fluorophenyl)-2-oxo-1-p-tolyl-ethyl]-4-methyl-3-oxo-pentanoic acid phenylamide ¹H NMR(CDCl₃): δ 1.16 (d, J=9Hz, 3H), 1.21 (d, J-9Hz, 3H), 2.25 (s, 3H), 3.00 (sep, J=6Hz, 1H), 4.50 (d, J=12Hz, 1H), 5.31 (d, J=12Hz, 1H), 6.95-7.35 (m, 11H), 0.36-7.55 (m, 1H), 7.92-8.07 (m, 2H).MS (+ve ion mode): m/z 433 [M+1] Yield: 78%

2-[2-(4-Fluorophenyl)-2-oxo-1-(4-trifluoromethylphenyl)-ethyl]-4-methyl-3-oxo-pentanoic acid phenylamide ¹H NMR(CDCl₃): δ 1.16 (d, J=6.8Hz, 3H), 1.24 (d, J=7.7Hz, 3H), 2.99 (Sep, 3=6.5Hz, 1H), 4.52 (d, J=10.6Hz, 1H),5.45 (d, J=10.7Hz, 1H), 6.95-7.20 (M, 4h), 7.21-7.37 (M, 4h), 7.42 (D, J=8Hz, 2H), 7.53 (d, J=7.8Hz, 2H), 7.95-8.08 (m, 2H) MS (+ve ion mode) m/z 486 [M+1] Yield: 52%

2-Cyclopropanecarbonyl-4-(4-fluorophenyl)-4-oxo-3,N-diphenyl-butyramide

¹H NMR(CDCl₃, 300 MHz): δ 0.86-1.13 (m, 4H), 2.17-2.20 (m, 1H), 4.60 (d, J=10.8Hz, 1H), 5.42 (d, J=10.8Hz, 1H), 6.88-7.47 (m, 12H), 7.98-8.03 (m, 2H).MS (+ve ion mode): m/z 416 (M⁺+1) Yield: 73%

Step 4: Preparation of Pyrrole (Formula X)

A mixture of Diketone (1 equiv), amine (1.00, equiv) and pivalic acid (1.03 equiv) in toluene was refluxed and water trapped using Dean Stark trap. After the completion of reaction, solvents were removed and residue dissolved in ethyl acetate. The organic layer was washed in saturated sodium bicarbonate, water, dried over anhydrous sodium sulphate, concentrated on rotavapor and residue purified on column (silica gel, 100-200 mesh).

The following intermediates were prepared following above general procedure:

(4R,6R)-(6-{2-[2,3-Di-(4-Fluorophenyl)-5-isopropyl-4-phenylcarbamoyl-pyrrol-1-yl]-ethyl}-2,2-dimethyl-[1,3]dioxan-4-yl)-acetic acid tert-butyl ester. MS (+ve ion mode): m/z=674 [M⁺+1]

(4R,6R)-(6-{2-[3-(2,4-Difluorophenyl)-2-(4-fluoro-phenyl)-5-isopropyl-4-phenyl carbamoyl-pyrrol-1-yl]-ethyl}-2,2-dimethyl-[1,3]dioxan-4-yl)-acetic acid tert-butyl ester

¹H NMR(CDCl₃): δ 1.30 (s, 3H), 1.36 (s, 3H), 1.43 (s, 9H), 1.51 (d, J=6Hz, 6H), 1.66 (brs, 2H), 2.20-2.50 (m, 2H), 3.48 (Sep, J=6.9Hz, 1H), 3.65-3.88 (m, 1H), 4.03-4.25 (m, 2H), 6.71 (t, J=8.4Hz, 2H), 6.96-7.35 (m, 12H) MS (+ve ion mode): m/z 692 [M⁺+!] Yield: 64%

(4R,6R)-(6-{2-[2-(4-Fluorophenyl)-5-isopropyl-4-phenylcarbamoyl-3-(4-trifluoromethylphenyl)-pyrrol-1-yl]-ethyl}-2,2-dimethyl-[1,3]dioxan-4-yl)-acetic acid tert-butyl ester

¹H NMR(CDCl₃): δ 1.30 (s, 3H), 1.37 (s, 3H), 1.37 (s, 3H), 1.44 (s, 9H), 1.52 (d, J=6.9Hz, 6H), 1.60-1.80 (m, 4H), 2.20-2.43 (m, 2H), 3.43 (Sep, J=6.9Hz, 1H), 3.67-3.87 (m, 2H), 4.00-4.28 (m, 2H), 6.77 (s, 1H), 6.99-7.35 (m, 11H), 7.41 (d, J=8.1Hz, 2H) MS (+ve ion mode): m/z 724 [M+1] Yield: 48%

(4R,6R)- (6-{2-[3-(4-Cyanophenylcarbamoyl)-5-(4-fluorophenyl)-2-isopropyl-4-phenylpyrrol-1-yl]-ethyl}-2,2-dimethyl-[1,3]dioxan-4-yl)-acetic acid tert-butyl ester

¹H NMR(CDCl₃): δ 1.30 (s, 3H), 1.36 (s, 3H), 1.43 (s, 9H), 1.53 (d, J=9Hz, 6H), 1.63 (brs, 2H), 2.15-2.48 (m, 2H), 3.58-3.90 (m, 3H), 4.05-4.27 (m, 2H), 6.95-7.28 (m, 12H), 7.43 (d, J=9Hz, 2H) MS (+n ve ion mode): m/z 680 [M+1] Yield: 56%

(4R,6R)-(6-{2-[2-Cyclopropyl-5-(4-fluorophenyl)-4-phenyl-3-phenylcarbamoyl-pyrrol-1-yl]ethyl}-2,2-dimethyl-[1,3]dioxan-4-yl)-acetic acid tert-butyl ester

¹H NMR(CDCl₃, 300 MHz): δ 0.77 (brs, 2H), 0.99-1.12 (m, 3H), 1.20 (s, 3H), 1.32 (s, 3H), 1.42 (s, 9H), 1.51-1.57 (m, 2H), 1.85-2.00 (m, 1H), 2.21 (dd, J=15 & 6Hz, 1H), 2.34 (dd, J=15 & 6Hz, 1H), 3.62 (brs, 1H), 4.12-4.22 (m, 3H), 6.88-7.36 (m, 14H).MS (+ve ion mode): m/z 653 (M⁺+1) Yield: 65%

(4R,6R)-(6-{2-[2-(4-Fluorophenyl)-5-isopropyl-4-phenylcarbamoyl-3-p-tolyl-pyrrol-1-yl]ethyl}-2,2-dimethyl-[1,3]dioxan-4-yl)-acetic acid tert-butyl ester ¹H NMR(CDCl₃): δ 1.30 (s, 3H), 1.36 (s, 3H), 1.43 (s, 9H), 1.52 (d, J=7.1Hz, 6H), 1.60 (brs, 4H), 2.20-2.45 (m, 5H), 3.53-3.90 (m, 3H), 4.00-4.25 (m, 2H), 6.90-7.20 (m, 13H) MS (+ve ion mode): m/z 670 [M+1] Yield: 35%

(4R,6R)-(6-{2-[2-(3,4-Difluorophenyl)-5-isopropyl-3-phenyl-4-phenylcarbamoyl-pyrrol-1yl]-ethyl}-2,2-dimethyl-[1,3]dioxan-4-yl)-acetic acid tert-butyl ester

¹H NMR(CDCl₃, 300 MHz): δ 1.05-1.13 (m, 2H), 1.29 (s, 3H), 1.39 (s, 3H), 1.44 (s, 9H), 1.51 (d, J=6Hz, 6H), 1.65-1.69 (m, 2H), 2.27 (dd, J=15 and 6hz, 1H), 2.36 (dd, J=15.8 & 6+12, 1H), 3.45-3.60 (m, 1H), 3.63-3.82 (m, 2H), 4.07-4.21 (m, 2H), 6.84-7.25 (m, 13H) MS (+ve ion mode): m/z 673 (M⁺+1) Yield: 44%

(4R,6R)-(6-{2-[2-(2,4-Difluorophenyl)-5-isopropyl-3-phenyl-4-phenylcarbamoyl-pyrrol-1-yl]-ethyl}-2,2-dimethyl-[1,3]dioxan-4-yl)-acetic acid tert-butyl ester

¹H NMR(CDCl₃, 300 MHz): δ 0.88-0.95 (m, 1H), 1.28 (s, 3H0, 1.38 (s, 31.43 (s, 9H), 1.54 (d, J=6Hz, 6H), 1.58-1.63 (m, 2H), 2.23 (dd, J=15 & 6Hz, 1H), 2.33 (dd, J=15 & 6Hz, 1H), 3.59-4.18 (m), 6.77-6.88 (m, 3H) MS (+ve ion mode): m/z 673 (M⁺+1)Yield: 43%

(4R,6R)-(6-{2-[3-(2,4-Difluorophenylcarbamoyl)-5-(4-fluorophenyl)-2-isopropyl-4-phenyl-pyrrol-1-yl]-ethyl}-2,2-dimethyl-[1,3]dioxan-4-yl)-acetic acid tert-butyl ester

¹H NMR(CDCl₃, 300 MHz): δ 1.03-1.11 (m, 2H), 1.3 (s, 3H), 1.37 (s, 3H), 1.43 (s, 9H), 1.55 (d, J=8.4Hz, 6H), 1.61-1.77 (m, 2H), 2.23 (dd, J=15.3Hz+6.3Hz), 2.39 (dd, J=15.3Hz+6.3Hz, H), 3.54-3.69 (m, 2H), 3.75-3.95 (m, 1H), 4.07-4.25 (m, 2H), 6.97-7.02 (m, 2H), 7.12-7.22 (m, 9H), 7.41 (d, J=8.4Hz, 2H) MS (+ve ion mode): m/z 723.3 [M⁺+1] Yield: 58%

(4R,6R)-(6-{2-[2-(4-Fluorophenyl)-4-(4-fluorophenylcarbamoyl)-5-isopropyl-3-phenyl-pyrrol-1-yl]-ethyl}-2,2-dimethyl-[1,3]dioxan-4-yl)-acetic acid tert-butyl ester

¹H NMR(CDCl₃): δ 0.9801.20 (m, 2H), 1.30 (s, 3H), 1.36 (s, 3H), 1.43 (s, 9H), 1.52 (d, J=7.1Hz, 6H), 1.60-1.73 (m, 2H), 2.18-2.42 (m, 2H), 3.49-3.60 (m, 1H), 3.69-3.79 (m, 1H), 3.80-3.90 (m, 1H), 3.97-4.23 (m, 2H), 6.75-7.31 (m, 13H) MS (+ve ion mode): m/z 673 [M+1] Yield: 47%

(4R,6R)-(6-{2-[2-(4-Fluorophenyl)-5-isopropyl-3-phenyl-4-(pyridin-4-ylcarbamoyl)-pyrrol-1yl]-ethyl}-2,2-dimethyl-[1,3]dioxan-4-yl)-acetic acid tert-butyl ester

¹H NMR(CDCl₃, 300 MHz): δ 0.95-1.12 (m, 2H), 1.28 (s, 3H), 1.38 (s, 3H), 1.43 (s, 3H), 1.52 (d, J=7.2Hz, 6H), 1.6-1.75 (m, 1H), 2.23 (dd, J=15H, 6.0Hz, 1H), 2.25-2.35 (m, 1H), 3.61-3.8 (m, 2H), 3.85-3.9 (m, 1H), 4.11-4.23 (m, 2H), 6.94-7.02 (m, 4H), 7.14-7.25 (m, 4H), 8.3 (d, J=6.0Hz, 2H) MS (+ve ion): m/z 656.3 [M⁺+1] Yield: 50%

(4R,6R)-(6-{2-[2-(4-Fluorophenyl)-5-isopropyl-3-phenyl-4-(pyridin-3-ylcarbamoyl)-pyrrol-1-yl]-ethyl}-2,2-dimethyl-[1,3]dioxan-4-yl)-acetic acid tert-butyl ester

¹H NMR(CDCl₃, 300 MHz): δ 0.9-1.2 (m, 2H), 1.3 (s, 3H), 1.36 (s, 3H), 1.43 (s, 9H), 1.53 (d, J=60Hz, 6H), 1.66 (brs, 2H), 2.23 (dd, J=15.0 & 6.0Hz, 1H), 2.38 (dd, J=15.0+6.0Hz, 1H), 3.61-3.69 (m, 2H), 3.8-3.9 (m, 1H), 4.09-4.17 (m, 2H), 6.87 (brs, 1H), 6.99 (t, J=9.0, 2H), 7.15-7.21 (m, 8H), 7.75 (brs, 1H), 7.95 (d, J=6.0Hz, 1H), 8.20 (d, J=3.0Hz, 1H) MS (+ve ion): m/z 656.5 [M⁺+1] Yield: 46%

(4R,6R)-(6-{2-[2-(4-Fluorophenyl)-4-(3-fluorophenylcarbamoyl)-5-isopropyl-3-phenyl-pyrrol-1-yl]-ethyl}-2,2-dimethyl-[1,3]dioxan-4-yl)-acetic acid tert-butyl ester

MS (+ve ion mode): m/z 673 [M⁺+1] Yield: 38%

Step 5: Preparation of Hemi Calcium Salt of Formula I

(a) To a solution of XI in methanol and tetrahydrofuran (1:1) was added 1N hydrochloric acid (3 equiv) and the mixture stirred at an ambient temperature. After the complete hydrolysis of ketal, reaction mixture was cooled to 0° C. and sodium hydroxide pellet (6 equiv) were added. The reaction was then allowed to stir at an ambient temperature. At the end of ester hydrolysis, solvents were removed and residue dissolved in water; aqueous layer was washed with ether, and neutralized with IN hydrochloric acid. Organics were extracted into ethyl acetate, and concentrated. The residue was then purified on column (silica gel 100-200 mesh).

(b) To an aqueous solution of sodium salt of acid (prepared by adding 1 equivalent 1N sodium hydroxide solution) was added dropwise an aqueous solution (1M) of calcium acetate (0.55 equiv). A little white precipitate was obtained which was filtered off and washed with copious amout of water, dried in vacuo.

The following compounds were prepared following above general procedure:

(3R,5R)-7-[2-(4-Fluorophenyl)-5-isopropyl-3-phenyl-4-[(3-fluorophenylamino) carbonyl]-1H-pyrrol-3,5-dihydroxy 1-heptanoic acid calcium salt(Compound No. 3),

1H NMR(DMSO-d₆, 300 MHz): δ 1.21 (brs-2H), 1.37 (brs, 6H), 1.53 (brs, 2H), (brs, 1H), 2.00 (brs, 1H), 3.25 (brs, 1H), 3.38 9brs, 1H), 3.70 (brs, 2H), 3.94 (brs, 1H), 6.81 (brs, 1H), 7.06 (brs, 4H), 7.23 (brs, 6H), 7.52 (brs, 2H), 10.11 (s, 1H, d₂O exchanged) MS (+ve ion mode): m/z 578 [acid+1] Yield: 32%

m.pt: 224.5-229° C.

(3R,5R)-7-[2-(4-Fluorophenyl)-5-isopropyl-3-phenyl-4-[(4-fluorophenylamino) carbonyl]-1H-pyrrol-3,5-dihydroxy-1-heptanoic acid calcium salt(Compound No. 4),

¹H NMR(DMSO-d₆): δ 1.23 (brs, 2H), 1.37 (d, J=5.7Hz, 6H), 1.56 (brs, 2H), 1.88-2.10 (m, 2H), 3.52 (brs, 1H), 3.73 (brs, 2H), 3.95 (brs, 1H), 7.06 9brs, 1H), 7.12-7.38 (brm, 4H), 7.53 9brs, 2H), 9.90 (s, 1H) d₂O exchange MS (+ve ion mode): m/z 577 [Acid+1] Yield: 18%

m.pt. 196.4-201.6° C.

(3R,5R)-7-[2-(4-Fluorophenyl)-5-isopropyl-3-phenyl-4-[(pyridin-3-yl-amino)carbonyl]-1H-pyrrol-3,5-dihydroxy-1-heptanoic acid calcium salt (Compound No. 6),

¹H NMR(DMSO-d₆, 300 MHz): δ 1.24 (brs, 2H), 1.37 (d, J=6.0Hz, 6H), 1.5-1.70 (m, 2H), 1.94 (dd, J=15.0 & 6.0H, 1H), 2.05 (dd, J=12Hz & 3.0Hz, 1H), 3.51 (brs, 2H), 3.74 (brs, 2H), 3.90-4.20 (m, 1H), 4.6-4.9 (brs, 1H (D₂0 exchange)), 7.0-7.11 (m, 5H), 7.16-7.25 (m, 5H), 7.95 (d, J=9.0Hz, 1H), 8.19 (d, J=6.0Hz, 1H), 8.6 s, 1H), 10.03 (s, 1H) MS (+ve ion mode): m/z 560.4 [Acid+1] Yield: 22%

(3R,5R)-7-[2-(4-Fluorophenyl)-5-isopropyl-3-phenyl-4-[(pyridin-4-yl-amino)carbonyl]-1H-pyrrol-3,5-dihydroxy-1-heptanoic acid calcium salt(Compound No. 7),

¹H NMR(DMSO-d₆, 300 MHz): δ 1.24 (brs, 2H), 1.36 (d, J=6.0Hz, 6H), 15-175 (m, 2H), 192 (dd, J=15.0 & 6.0H, 1H), 2.0-212 (m, 1H), 3.1-3.55 (m, 2H), 3.74 (brs, 2H), 4.0 (brs, 1H), 6.87-7.07 (m, 5H), 716-7.25 (m, 4H), 7.48 (d, J=60H, 2H), 8.32 (d, J=3.0Hz, 2H), 10.23 (s, 1H) MS(+ve ion): m/z 560.1 [acid+1]+Yield: 7%

Melting Point (° C.): 212.9-215.2° C.

(3R,5R)-7-[2-(4-Fluorophenyl)-5-isopropyl-3-phenyl-4-[(4-cyanophenylamino) carbonyl]-1H-pyrrol-3,5-dihydroxy-1-heptanoic acid calcium salt (Compound No. 9),

¹H NMR (DMSO-d₆): δ 1.12-1.42 (brs, 8H), 1.55 (m, 2H), 1.84-2.10 (m, 2H), 3.52 (brs, 1H), 3.75 (brs, 2H), 3.96 (brs, 1H), 6.95-7.30 (m, 9), 7.70 (brs, 4H), 10.28 (s, 1H, d₂O exchanged) MS (+ve ion mode): m/z 584 [Acid+1] Yield: 30.05%

m.pt: 209-249° C.

(3R,5R)-7-[2-(4-Fluorophenyl)-5-isopropyl-3-phenyl-4-[(2,4-difluorophenylamino) carbonyl]-1H-pyrrol-3,5-dihydroxy-1-heptanoic acid calcium salt (Compound No. 10),

¹H NMR(DMSO-d₆, 300 MHz): δ 1.23 (brs, 2H), 1.36 (d, J=6Hz, 6H), 1.50-1.75 (m, 2H), 1.90-2.20 (m, 2H), 3.51 (brs, 1H), 3.71 (brs, 2H), 3.95 (brs, 1H), 7.06 (brs, 5H), 7.15-7.24 (m, 4H), 7.52 (brs, 2H), 9.93 (5, 1H) MS (+ve ion mode): m/z 595 (acid+1) Yield: 34.02%

Melting Point=249.5-272.8° C.

(3R,5R)-7-[2-(2,4-Difluorophenyl)-5-isopropyl-3-phenyl-4-[(phenylamino)carbonyl]-1H-pyrrol-3,5-dihydroxy-1-heptanoic acid calcium salt (Compound No. 12),

¹H NMR(DMSO-d₆, 300 MHz): δ 1.14 (brs, 2H), 1.30 (d, J=6Hz, 6H), 1.44 (brs, 2H), 1.82 (dd, J=14.1 & 7.2Hz, 1H), 1.94-1.98 (m), 3.17 (sept, J=7.2Hz, 1H), 3.45 (brs, 1H), 3.74 (brs, 1H), 3.82 (brs, 1H), 3.86 (brs, 1H), 6.89-7.07 (m, 7H), 7.12-7.28 (m, 4H), 7.46 (d, J=7.8Hz, 2H) MS (+ve ion mode): m/z 577 (acid+1)+Yield: 29%

Melting point ° C.=203.6-217.4° C.

(3R,5R)-7-[2-(3,4-Difluorophenyl)-5-isopropyl-3-phenyl-4-[(phenylamino)carbonyl]-1H-pyrrol-3,5-dihydroxy-1-heptanoic acid calcium salt (Compound No. 13),

¹H NMR(DMSO-d₆, 300 MHz): δ 1.21-1.26 (m, 1H), 1.36 d, J=6.6Hz, 6H), 1.32-1.45 (m, 3H), 191 (dd, J=15 & 6Hz, 1H), 2.06 (dd, J=14.7 & 4Hz, 1H), 3.21-3.54 (m, 2H), 3.57-4.02 (m, 3H), 6.96-7.13 (m, 7H), 7.19-7.27 (m, 3H), 7.39-7.52 (m, 3H), 9.87 (s, 1H). MS (+ve ion mode): m/z 576 (Acid+1) Yield: 66%

m.p.=169-231° C.

(3R,5R)-7-[2-(4-Fluorophenyl)-5-isopropyl-3-(2,4-difluorophenyl)-4-[(phenylamino) carbonyl]-1H-pyrrol-3,5-dihydroxy-1-heptanoic acid calcium salt (Compound No. 15),

¹H NMR(DMSO-d₆): δ 1.27 (brs, 2H), 1.38 (d, J=5.86Hz, 6H), 1.57 (brs, 2H), 1.88-2.13 (m, H), 3.27 (brs, 1H), 3.55 (brs, 1H) (brs, 2H) 4.00 (brs, 1H), 6.83-7.03 (m, 3H), 7.17 (brs, 7H), 7.45 (brs, 2H), 9.63 (s, 1H, D₂O exchanged) MS (+ve ion mode): m/z 596 [Acid+1] Yield 31%

m.p. 172.8-221.1° C.

(3R,5R)-7-[2,3-Di-(4-fluorophenyl)-5-isopropyl-4-[(phenylamino)carbonyl]-1H-pyrrol-3,5-dihydroxy-1-heptanoic acid calcium salt(Compound No. 16),

¹H NMR(DMSO-D₆): δ 1.09 (brs, 2h), 1.21 (D, 5=6.5Hz, 6H), 1.57 (brs, 2H), 1.85-2.20 (m, 2H), 3.22 (brs, 1H), 3.37 (brs, 1H) (brs, 2H) 3.76 (brs, 1H), 6.85-7.12 (m, 5H), 7.13-7.35 (m, 6H), 7.48-7.65 (m, 2H), 9.87 (s, 1H, d₂O exchanges) MS (+ve ion mode): m/z 578 [Acid+1] m.p. 175.7-212.4° C. yield 20%

(3R,5R)-7-[2-(4-Fluorophenyl)-5-isopropyl-3-(4-methylphenyl)-4-[(phenylamino)carbonyl]-1H-pyrrol-3,5-dihydroxy-1-heptanoic acid calcium salt (Compound No. 17),

¹H NMR(CDCl₃): δ 1.24 (brs, 2H), 1.36 (d, 6H, J=9Hz), 1.56 (brs, 2H), 1.89-2.22 (m, 5H), 3.21 (brs, 2H), 3.52 (bras, 1H), 3.74 (bras, 2H), 3.94 (brs, 1H), 6.85-7.05 (m, 5H), 7.15-7.30 (m, 6H), 7.54 (d, J=6Hz, 2H), 9.87 (s, 1H, d₂O exchanged) MS (+ve ion mode): m/z 574 [Acid+1] Yield: 40%

m.pt. 195-217.4° C.

(3R,5R)-7-[2-(4-Fluorophenyl)-5-isopropyl-3-(4-trifluoromethylphenyl)-4-[(phenylamino)carbonyl]-1H-pyrrol-3,5-dihydroxy-1-heptanoic acid calcium salt (Compound No. 18),

¹H NMR(DMSO): δ 1.26 (brs, 2h), 1.38 (D, j=6Hz, 6H), 1.62 (brs, 2H), 1.95-220 (m, 2H), 3.25 (brs, 1H), 3.54 (brs, 1H) (brs, 2H), 4.05 (brs, 1H), 7.0 (t, J=7.1Hz, 1H), 7.25 (brs, 8H), 7.45 (d, J=7.8Hz, 2H), 7.54 (d, J=7.6Hz, 2H), 10.02 (s, 1H, d₂O exchanged) MS (+ve ion mode): m/z 628 [Acid+1] Yield 17%, m.p 174.3-221.3° C.

(3R,5R)-7-[2-(4-Fluorophenyl)-5-cyclopropyl-3-phenyl-4-[(phenylamino)carbonyl]-1H-pyrrol-3,5-dihydroxy-1-heptanoic acid calcium salt (Compound No. 19),

¹H NMR(DMSO-d₆, 300 MHz): δ 0.63 (brs, 2H), 0.82 (brs, 2H), 1.15-1.44 (m, 2H), 1.88-2.10 (m, 3H), 3.51 (brs, 1H), 3.72 (brs, 1H), 3.90-4.11 (m, 2H), 6.99-7.09 (m, 6H), 7.14-7.32 (m, 6H), 7.60 (d, J=6Hz, 2H), 10.04 (s, 1H).

MS (+ve ion mode): m/z 557 (M⁺+1)

Yield=26%

m.p.=160-230° C.

Scheme II

Preparation of Compound of Formula XII

To a solution of a compound of Formula XI (4.5 mmoles) in toluene (15 ml) was added a compound of Formula V (4.9 mmoles), piperidine (0.02 ml) and acetic acid (0.054 ml). The mixture was heated at reflux with azeotropic removal of water for 4 to 6 hours. The reaction mixture was concentrated and residue extracted in dichloromethane. Organic layer washed with 1N hydrochloric acid solution, sodium bicarbonate solution, brine, dried over anhydrous sodium sulphate and concentrated. The crude product was purified on column (silica gel, 100-200 mesh, 2% EtOAc-hexane)

¹H NMR(CDCl₃, 300 MHz): δ 1.02 (d, J=6.9Hz, 6H), 2.65 (sept, J=7.2Hz, 1H), 5.26 (s, 2H), 7.25 (s, 2H), 7.25 (brs, 10H), 7.81 (s, 1H). ). isomer 2: ¹H NMR(CDCl₃, 300 MHz): δ 1.02 (d, J=6.9 Hz, 6H), 2.65 (sept, J=6.9 Hz, 1H), 5.27 (s, 2H), 7.36 (brs, 10H), 7.82 (s, 1H).MS (+ve ion mode): m/z 309 (M⁺+1); Yield: 70%

Preparation of Compound of Formula XIII

Compound of Formula XII (6.49 mmoles), compound of Formula VII (7.14 mmoles), thiazolium bromide (1.298 mmoles) triethylamine (6.49 mmoles) and ethanol (0.6 ml) were placed in a 30 ml vial, flushed with argon and the sealed vial properly. The reaction mixture stirred at 70° C. for 12 to 15 hours. To the reaction mixture was added ethyl acetate, washed with water, 6N hydrochloric acid, again with water and brine, dried over anhydrous sodium sulphate, and concentrated to give crude product. The crude product was purified on column (silica gel 100-200 mesh) using 7% ethyl acetate in hexane.

¹H NMR(CDCl₃, 300 MHz): (1:1 mixture of diastereomers) δ 0.48 (d, J=6.9Hz, 3H), 0.91 (d, J=6.6Hz, 3H), 1.07 (d, J=6.6Hz, 3H), 1.21 (d,J=6.9Hz, 3H), 2.30 (sept, J=6.6Hz, 1H), 2.82 (sept, 6.6Hz, 1H), 4.76 (d, J=14Hz, 1H), 4.77 (d, J=12.3Hz, 1H), 5.33 (d, J=11.1Hz, 1H), 5.35 (d, J=11.1Hz, 1H), 7.02 (t, J=8.4Hz, 6H), 7.22-7.29 (m, 8H), 7.75-7.99 (m, 4H) MS (+ve ion mode): m/z 433 (M⁺+1). Yield: 72%

Preparation of Compound of Formula XIV

To a solution of Formula XIII (4.62 mmoles) in heptane:toluene:tetrahydrofuran (4:1:1) was added a compound of Formula IX (6.99 mmoles) and pivalic acid (4.768 mmoles). The mixture was refluxed with azeotropic removal of water for 22 to 25 hours. The reaction mixture was concentrated, ethyl acetate was added, washed with sodium bicarbonate solution and brine, dried over anhydrous sodium sulphate and concentrated to give the crude product. The crude product was purified on column (silica gel, 100-200 mesh) using 7% ethyl acetate in hexane.

¹H NMR(CDCl₃, 300 MHz): δ 0.99-1.08 (m, 2H), 1.25 (s, 3H), 1.34 (s), 1.43 (s, 9H), 1.96 (d, J=6Hz, 6H), 1.58-1.63 (m, 2H), 2.21 (dd, J=158.6Hz, 1H), 2.37 (dd, J=15 & 9Hz, 1H), 3.51 (sept, J=6Hz), 3.65 (brs, 1H), 3.75-3.85 (m, 1H), 4.00-4.25 (m, 2H), 5.03 (s, 2H), 6.83-7.25 (m, 14H). MS (+ve ion mode): m/z 670 (M⁺+1). yield 74%

Preparation of Compound of Formula XV

To a solution of a compound of Formula XIV (0.8 g) in methanol: dioxan (2:8) mixture was added 10% palladium carbon (50% wet, 60% w/w). The resulting reaction mixture was hydrogenated at 40 psi for about 2.5 hours. After the reaction was over, the reaction mixture was passed through celite and the resulting solution was concentrated under vaccum to give the required product, which was used for the next step.

¹H NMR(CDCl₃, 300 MHz): δ 0.95-1.05 (m, 1H), 1.21-1.28 (m, 1H), 1.28 (s, 3H), 1.34 (s, 3H), 1.43 (s, 9H), 1.47 (d, J=7.1Hz), 1.59-1.65 (m, 2H), 2.22 (dd, J=15.2 & 6.1Hz, 1H), 2.35 (dd, J=15.2 & 6.1Hz, 1H), 3.61-3.66 (m, 2H), 3.67-3.86 (m, 1H), 4.00-4.15 (m, 2H), 6.95 (t, J=9Hz, 2H), 7.06-7.15 (m, 7H) MS (+ve ion mode): m/z 586 (M⁺+1) Yield 76%

Preparation of Compound of Formula X: path a

To a solution of a compound of Formula XV (1 equiv) in benzene at 0° C. under argon, oxalyl chloride (2.0 equiv) was added dropwise. After the evolution of gas had ceased, the reaction mixture was heated on oil bath at 70° C. for 2 hours. Reaction mixture was evaporated to dryness. The residue was dissolved in benzene (dry) and added at ambient temperature to a solution of amine of formula III (1.1 equiv.) in benzene. The reaction mixture was then heated at 70° C. till completion of reaction. Volatiles were removed in vacuo & the residue was purified on column (silica gel, 100-200 mesh).

The following compounds were prepared following using the above general procedure:

(4R,6R)-(6-{2-[3-(2-Cyanophenylcarbamoyl)-5-(4-fluorophenyl)-2-isopropyl-4-phenyl pyrrol-1-yl]-ethyl}-2,2-dimethyl- [1,3]dioxan-4-yl)-acetic acid tert-butyl ester

¹H NMR(CDCl₃, 300 MHz): δ 1.0-1.15 (m, 1H), 1.30 (s, 3H), 1.44 (s, 9H), 50 (d, J=6.9Hz, 6H), 1.65-1.70 (m, 2H), 2.24 (dd, J=15.3Hz, +5.4Hz, 1H), 2.39 (dd, J=15.3Hz+6.6Hz, 1H), 3.46 (sep, 7.66Hz, 1H), 3.62-3.0 (m, 2H), 4.0-4.18 (m, 2H), 6.91-7.40 (m, 11H), 7.51 (t, J=80H, 1H), 8.43 (d, J=8.7Hz, 1H) MS (+ve ion): m/z 680.3 [M⁺+1] yield=54%

(4R,6R)-(6-{2-[2-(4-Fluorophenyl)-5-isopropyl-3-phenyl-4-(4-trifluoromethylphenyl carbamoyl) -pyrrol-1-yl]-ethyl}-2,2-dmethyl-[1,3]dioxan-4-yl)-acetic acid tert-butyl ester

¹H NMR(CDCl₃, 300 MHz): δ 1.03-1.11 (m, 2H), 1.3 (s, 3H), 1.37 (s, 3H), 1.43 (s, 9H), 1.55 (d, J=8.4Hz, 6H), 1.61-1.77 (m, 2H), 2.23 (dd, J=15.3Hz+6.3Hz), 2.39 (dd, J=15.3Hz+6.3Hz, H), 3.54-3.69 (m, 2H), 3.75-3.95 (m, 1H), 4.07-4.25 (m, 2H), 6.97-7.02 (m, 2H), 7.12-7.22 (m, 9H), 7.41 (d, J=8.4Hz, 2H) MS (+ve ion mode): m/z 723.3 [M⁺+1] yield=57%

Preparation of Compound of Formula X: path b

To a solution of XV (1 equiv.) in THF (dry) at −15 ° C. was added triethylamine (1 equiv.) followed by isobutyrylchloroformate (1 equiv.) and the reaction mixture stirred at −15° C. for 15 min. A solution of amine III (1 equiv.) in tetrahydrofuran was then added followed by para-toluene sulfonic acid (0.1 equiv.). The reaction mixture was heated at 55-60° C., overnight. After the completion of reaction, reaction mixture was diluted with dichloromethane, washed with 0.1N hydrochloric acid, sodium bicarbonate, brine, and the organic layer dried over sodium sulfate and concentrated in vacuo. The residue was purified by column chromatography (silica-gel, 100-200 mesh)

The following compound was prepared as per this protocol:

(4R,6R)-(6-{2-[2-(4-Fluorophenyl)-4-(2-fluorophenylcarbamoyl)-5-isopropyl-3-phenyl-pyrrol-1-yl]-ethyl}-2,2-dimethyl-[1,3]dioxan-4-yl)-acetic acid tert-butyl ester

¹H NMR(CDCl₃, 300 MHz): δ 1.03-1.10 (m, 2H), 1.3 (s, 3H), 1.36 (s, 3H), 1.43 (s, 9H), 1.51 (J, J=7.2Hz, 6H), 2.23 (dd, J=15 & 6Hz, 1H), 2.33 (dd, 5.15 & 6.9Hz, 1H), 3.54 (sept, J=7.2Hz, 1H), 3.67 (brs, 1H), 3.87-3.95 (m, 1H), 4.05-4.30 (m, 2H), 6.87-7.18 (, 14H), 8.36 (d, J=7.8Hz, 1H) MS (+ve ion mode): m/z 673 (M⁺+1) Yield 35%

Preparation of Hemi Calcium Salt of Formula I

To a solution of X in methanol and tetrahydrofuran (1:1) was added 1N hydrochloric acid (3 equiv) and the mixture stirred at an ambient temperature. After the complete hydrolysis of ketal, reaction mixture was cooled to 0° C. and sodium hydroxide pellet (6 equiv) were added. The reaction was then allowed to stir at an ambient temperature. At the end of ester hydrolysis, solvents were removed and the residue was dissolved in water; the aqueous layer was washed with ether, and neutralized with 1N hydrochloric acid. Organics were extracted into ethyl acetate, and concentrated. The residue was then purified on column (silica gel 100-200 mesh).

(b) To an aqueous solution of sodium salt of acid (prepared by adding 1 equivalent 1N sodium hydroxide solution) was added dropwise an aqueous solution (1M) of calcium acetate (0.55 equiv). A little white precipitate was obtained which was filtered off and washed with copious amout of water, dried in vacuo.

The following compounds were prepared following above general procedure:

(3R,5R)-7-[2-(4-Fluorophenyl)-5-isopropyl-3-phenyl-4-[(2-fluorophenylamino) carbonyl]-1H-pyrrol-3,5-dihydroxy-1-heptanoic acid calcium salt (Compound No. 2),

¹H NMR(DMSO-d₆, 300 MHz): δ 1.24 (brs, 2H), 1.42 (d, J=9Hz, 6H), 1.51-1.58 (m, 2H), 1.90 (dd, J=15 & 9Hz, 1H), 2.02-2.08 (m) 3.59 (brs, 1H), 3.73 (brs, 1H), 3.96 (brs, 1H), 7.11-7.29 (m, 1), 9.20 (s, 1H) MS (+ve ion mode): m/z 577 (Acid+1) Yield 53%

m.p: 165-176° C.

(3R,5R)-7-[2-(4-Fluorophenyl)-5-isopropyl-3-phenyl-4-[(2-cyanophenylamino)carbonyl]-1H-pyrrol-3,5-dihydroxy-1-heptanoic acid calcium salt (Compound No. 8),

¹H NMR(DMSO-300 MHz): δ 1.19-1.28 (m, 2H), 1.41 (d, J=6.0z, 6H), 1.54-1.62 (m, 2H), 1.88 (dd, J=1.50+9.0Hz, 1H), 2.04 (dd, J=15.0+3.0Hz, 1H), 3.22-3.68 (m, 2H), 3.7-3.78 (m, 2H), 3.96 (brs, 1H), 7.04-7.30 (m, 11H), 7.60 (t, J=9.0Hz), 7.72 (d, J=6.0Hz, 1H), 9.82 (s, 1H) MS (+ve ion): m/z 584.200 [Acid+1] Yield=13%

(3R,5R)-7-[2-(4-Fluorophenyl)-5-isopropyl-3-phenyl-4-[(4-trifluoromethylphenylamino) arbonyl]-1H-pyrrol-3,5-dihydroxy-1-heptanoic acid calcium salt (Compound No. 11),

¹H NMR(DMSO-d6, 300 MHz): δ 1.15-1.30 (m, 2H), 1.35 (d, J=6.0Hz, 6H), 1.57-1.73 (m, 2H, 1.85 (dd, J=15.0+6.0Hz, 1H), 2.0 (dd, J=15.0+3.0Hz, 1H), 3.43-3.57 (m, 2H), 3.68-3.73 (m, 2H), 3.78-4.16 (m, 1H), 6.99 (brs, 1H), 7.14-7.24 (m, 4H), 7.56 (d, J=9.0Hz, 2H), 7.11 (d, J=9.0Hz, 2H), 10.241 & 1H). MS (+ve ion mode ): m/z 627.3 [acid+1]

yield=21%

Melting Point=214.5-216° C.

Pharmacological Activity

The compounds of the invention are inhibitors of 3-hydroxy-3-methyl-glutanyl coenzyme A (HMG-CoA) reductase and thus are useful in inhibiting cholesterol biosynthesis and/or in lowering triglycerides.

The compounds described herein were screened in an in-vitro HMG-CoA reductase enzyme assay as described by Kubo et al., Endocrinology 120: 214, (1987) and Hellar, et al. Biochem and Biophys. Res. Comm., 50: 859, (1973).

HMG-CoA reductase is a rate-limiting enzyme in the cholesterol biosynthesis, catalyzing the following reaction.

[¹⁴C] HMG-CoA+2NADPH+2H⁺→[¹⁴C] mevanolate+CoA+2NADP⁺ microsomes,

utilizing 2.5 μM [¹⁴C] HMG-CoA as a substrate. The reaction was carried out in the presence of 100 mM KH₂PO₄, 20 mM G-6-P, 2.5 mM NADPH, 10 mM EDTA, 5 mM DTT and 1.4 G-6-P dehydrogenase, at 37° C. for 15 minutes and quantitating [¹⁴C] mevalonate as an end product. For IC₅₀ determinations, the compounds were dissolved in 1% dimethylsulfoxide and were preincubated with liver microsomes at 37° C. for 30 minutes. The IC₅₀ of the tested compounds are given in Table 2.

TABLE 2

| Compound No. | IC₅₀ (pM) |
|---|---|
| 2 | 17.7 |
| 3 | 23.9 |
| 4 | 9.8 |
| 5 | 19.5 |
| 12 | 12.3 |
| 13 | 6.25 |
| 15 | 12.2 |
| 16 | 34.5 |
| 17 | 34.0 |
| 18 | 12.6 |
| 19 | 12.4 |
| Atovastatin | 64.6 |
| Rosuvastatin | 38.5 |

We claim:

1. (3R,5R)-7-[2-(4-Fluorophenyl)-5-isopropyl-3-phenyl-4-[(N-methyl-N-phenyl amino)carbonyl]-pyrrol-1-yl]-3,5-dihydroxy-1-heptanoic acid, its lactone form, pharmaceutically acceptable salts, pharmaceutically acceptable solvates, N-oxide or polymorphs.

2. (3R,5R)-7-[2-(4-Fluorophenyl)-5-isopropyl-3-phenyl-4-[(2-fluorophenylamino) carbonyl]-pyrrol-1-yl]-3,5-dihydroxy-1-heptanoic acid, its lactone form, pharmaceutically acceptable salts, pharmaceutically acceptable solvates, N-oxide or polymorphs.

3. (3R,5R)-7-[2-(4-Fluorophenyl)-5-isopropyl-3-phenyl-4-[(3-fluorophenylamino) carbonyl]-pyrrol-1-yl]-3,5-dihydroxy 1-heptanoic acid, its lactone form, pharmaceutically acceptable salts, pharmaceutically acceptable solvates, N-oxide or polymorphs.

4. (3R,5R)-7-[2-(4-Fluorophenyl)-5-isopropyl-3-phenyl-4-[(4-fluorophenylamino) carbonyl]-pyrrol-1-yl]-3,5-dihydroxy-1-heptanoic acid, its lactone form, pharmaceutically acceptable salts, pharmaceutically acceptable solvates, N-oxide or polymorphs.

5. (3R,5R)-7-[2-(4-Fluorophenyl)-5-isopropyl-3-phenyl-4-[(pyridin-2-yl-amino) carbonyl]-pyrrol-1-yl]-3,5-dihydroxy-1-heptanoic acid, its lactone form, pharmaceutically acceptable salts, pharmaceutically acceptable solvates, N-oxide or polymorphs.

6. (3R,5R)-7-[2-(4-Fluorophenyl)-5-isopropyl-3-phenyl-4-[(pyridin-3-yl-amino) carbonyl]-pyrrol-1-yl]-3,5-dihydroxy-1-heptanoic acid, its lactone form, pharmaceutically acceptable salts, pharmaceutically acceptable solvates, N-oxide or polymorphs.

7. (3R,5R)-7-[2-(4-Fluorophenyl)-5-isopropyl-3-phenyl-4-[(pyridin-4-yl-amino) carbonyl]-pyrrol-1-yl]-3,5-dihydroxy-1-heptanoic acid, its lactone form, pharmaceutically acceptable salts, pharmaceutically acceptable solvates, N-oxide or polymorphs.

8. (3R,5R)-7-[2-(4-Fluorophenyl)-5-isopropyl-3-phenyl-4-[(2-cyanophenylamino) carbonyl]- pyrrol-1-yl]-3,5-dihydroxy-1-heptanoic acid, its lactone form, pharmaceutically acceptable salts, pharmaceutically acceptable solvates, N-oxide or polymorphs.

9. (3R,5R)-7-[2-(4-Fluorophenyl)-5-isopropyl-3-phenyl-4-[(4-cyanophenylamino) carbonyl]-pyrrol-1-yl]-3,5-dihydroxy-1-heptanoic acid, its lactone form, pharmaceutically acceptable salts, pharmaceutically acceptable solvates, N-oxide or polymorphs.

10. (3R,5R)-7-[2-(4-Fluorophenyl)-5-isopropyl-3-phenyl-4-[(2,4-difluorophenyl amino)carbonyl]-pyrrol-1-yl]-3,5-dihydroxy-1-heptanoic acid, its lactone form, pharmaceutically acceptable salts, pharmaceutically acceptable solvates, N-oxide or polymorphs.

11. (3R,5R)-7-[2-(4-Fluorophenyl)-5-isopropyl-3-phenyl-4-[(4-trifluoromethyl phenylamino)carbonyl]-pyrrol-1-yl]-3,5-dihydroxy-1-heptanoic acid, its lactone form, pharmaceutically acceptable salts, pharmaceutically acceptable solvates, N-oxide or polymorphs.

12. (3R,5R)-7-[2-(2,4-Difluorophenyl)-5-isopropyl-3-phenyl-4-[(phenylamino) carbonyl]-pyrrol-1-yl]-3,5-dihydroxy-1-heptanoic acid, its lactone form, pharmaceutically acceptable salts, pharmaceutically acceptable solvates, N-oxide or polymorphs.

13. (3R,5R)-7-[2-(3,4-Difluorophenyl)-5-isopropyl-3-phenyl-4-[(phenylamino) carbonyl]-pyrrol-1-yl]-3,5-dihydroxy-1-heptanoic acid, its lactone form, pharmaceutically acceptable salts, pharmaceutically acceptable solvates, N-oxide or polymorphs.

14. (3R,5R)-7-[2-Cyclohexyl-5-isopropyl-3-phenyl-4-[(phenylamino)carbonyl]-pyrrol-1-yl]-3,5-dihydroxy-1-heptanoic acid, its lactone form, pharmaceutically acceptable salts, pharmaceutically acceptable solvates, N-oxide or polymorphs.

15. (3R,5R)-7-[2-(4-Fluorophenyl)-5-isopropyl-3-(2,4-difluorophenyl)-4-[(phenylamino)carbonyl]-1H-pyrrol-3,5-dihydroxy-1-heptanoic acid, its lactone form, pharmaceutically acceptable salts, pharmaceutically acceptable solvates, N-oxide or polymorphs.

16. (3R,5R)-7-[2,3-Di-(4-fluorophenyl)-5-isopropyl-4-[(phenylamino)carbonyl]-pyrrol-1-yl]-3,5-dihydroxy-1-heptanoic acid, its lactone form, pharmaceutically acceptable salts, pharmaceutically acceptable solvates, N-oxide or polymorphs.

17. (3R,5R)-7-[2-(4-Fluorophenyl)-5-isopropyl-3-(4-methylphenyl)-4-[(phenylamino)carbonyl]-pyrrol-1-yl]-3,5-dihydroxy-1-heptanoic acid, its lactone form, pharmaceutically acceptable salts, pharmaceutically acceptable solvates, N-oxide or polymorphs.

18. (3R,5R)-7-[2-(4-Fluorophenyl)-5-isopropyl-3-(4-trifluoromethylphenyl)-4-[(phenylamino)carbonyl]-pyrrol-1-yl]-3,5-dihydroxy-1-heptanoic acid, its lactone form, pharmaceutically acceptable salts, pharmaceutically acceptable solvates, N-oxide or polymorphs.

19. (3R,5R)-7-[2-(4-Fluorophenyl)-5-cyclopropyl-3-phenyl-4-[(phenylamino) carbonyl]-pyrrol-1-yl]-3,5-dihydroxy-1-heptanoic acid, its lactone form, pharmaceutically acceptable salts, pharmaceutically acceptable solvates, N-oxide or polymorphs.

20. A pharmaceutically acceptable salt of a compound of any one of the preceding claims. The salt of claim 20, wherein the the salts selected from the group comprising of lithium, sodium, potassium, calcium, magnesium, zinc, aluminium, amino acid, ammonium, monoalkyl ammonium, dialkyl ammonium, trialkyl ammonium and N-methyl glucamine.

21. The pharmaceutically acceptable salt of claim 20 wherein the salt is monosodium salt.

22. The pharmaceutically acceptable salt of claim 20 wherein the salt is monopotassium salt.

23. The pharmaceutically acceptable salt of claim 20 where the salt is hemicalcium salt.

24. The pharmaceutically acceptable salt of claim 20 where the salt is hemimagnesium salt.

25. The pharmaceutically acceptable salt of claim 20 where the salt is hemizinc salt.

26. The pharmaceutically acceptable salt of claim 20 where the salt is N-methyl glucamine salt.

27. A pharmaceutical composition comprising a therapeutically effective amount of a compound of any one of the preceding claims together with a pharmaceutically acceptable carrier, excipient or diluent.

28. A method for treating or preventing a mammal suffering from cholesterol-related disease, diabetes and related disease, cerebrovascular disease or cardiovascular disease, comprising administering to the said mammal, a therapeutically effective amount of a compound of any one of the preceding claims 1-26.

29. A method for treating or preventing a mammal suffering from cholesterol-related disease, diabetes and related disease, cerebrovascular disease or cardiovascular disease, comprising administering to the said mammal, a therapeutically effective amount of a composition according to claim 27.

30. The method according to claim 28 or 29 wherein the disease is selected from the group comprising of arteriosclerosis, atherosclerosis, hyperlipidemia, hypercholesterolemia, hypertriglyceridemia, hypertension, stroke, ischemia, endothellium, dysfunctions, peripheral vascular disease, peripheral arterial disease, coronary heart disease, myocardial infarction, cerebral infarction, myocardial microvascular disease, dementia, Alzheimer's disease, osteoporosis and/or osteopenia, angina or restenosis.

31. The method according to claim 30 wherein the disease is hyperlipidemia.

32. The method according to claim 30 wherein the disease is hypercholesterolemia.

33. The method according to claim 30 wherein the disease is hyperlipoproteinemia.

34. The method according to claim 30 wherein the disease is hypertriglyceridemia.

35. The method according to claim 30 wherein the disease is hypertension.

36. A process for the preparation of a compound of Formula I, 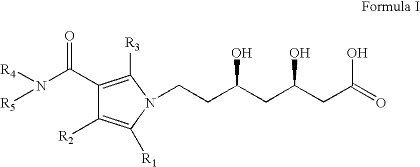
its pharmaceutically acceptable salts, pharmaceutically acceptable solvates, polymorphs or N-oxide wherein
R₁ is 4-fluorophenyl, 2,4-difluorophenyl, 3,4-difluorophenyl or cyclohexyl;
R₂ is phenyl, 4-fluorophenyl, 2,4-difluorophenyl, 4-methylphenyl or 4-trifluoromethylphenyl;
R₃ is isopropyl or cyclopropyl;
R₄ is hydrogen or methyl;
R₅ is phenyl, 2- fluorophenyl 3- fluorophenyl, 4-fluorophenyl, 2- pyridyl, 3- pyridyl, 4-pyridyl, 2-cyanophenyl, 4-cyanophenyl, 2,4-difluorophenyl, or 4-trifluoromethylphenyl, with the proviso that simultaneously R₁, R₂, R₃, R₄ and R₅ can not be respectively, 4-fluorophenyl, phenyl, isopropyl, hydrogen and phenyl comprising reacting a compound of Formula II with a compound of Formula III to give a compound of Formula IV, 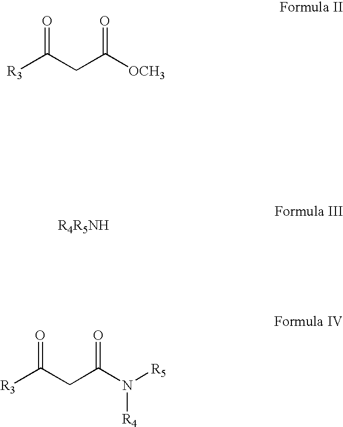
which on treatment with an aldehyde of Formula V gives a compound of Formula VI, 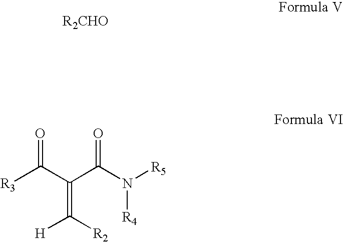
which on treatment with an aldehyde of Formula VII gives a compound of Formula VIII, 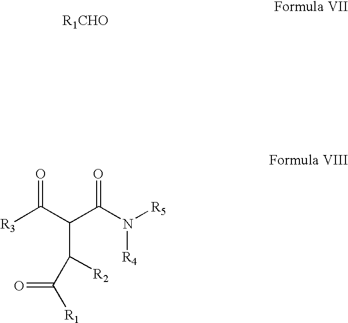
which on treatment with a compound of Formula IX gives a compound of Formula X, 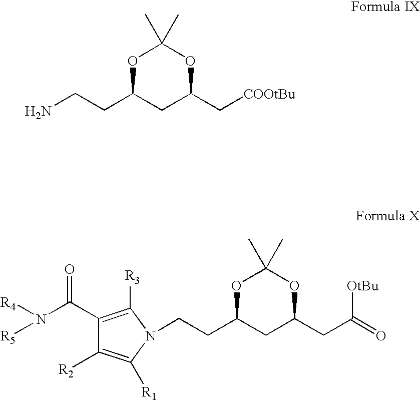
which on hydrolysis gives a compound of Formula I

37. The process according to claim 36 wherein the reaction of a compound of Formula I with a compound of Formula III to give a compound of Formula IV is carried out in a suitable solvent selected from the group comprising of xylene and toluene.

38. The process according to claim 37 is carried out in xylene.

39. The process according to claim 36 wherein the reaction of a compound of Formula II with a compound of Formula III is carried out in the presence of a suitable base selected from the group comprising of triethylamine, pyridine and 1,2-ethylenediamine.

40. The process according to claim 39 is carried out in the presence of 1,2-ethylenediamine.

41. The process according to claim 36 wherein the reaction of a compound of Formula IV with an aldehyde of Formula V to give a compound of Formula VI is carried out in a suitable solvent selected from the group comprising of hexane, heptane, dichloromethane and toluene.

42. The process according to claim 41 is carried out in hexane.

43. The process according to claim 36 wherein the reaction of a compound of Formula IV with an aldehyde of Formula V is carried out in the presence of an organic base selected from the group comprising of piperidine, pyridine and β-alanine and an organic acid selected from the group comprising of glacial acetic acid and benzoic acid.

44. The process according to claim 43 is carried out in the presence of β-alanine and glacial acetic acid.

45. The process according to claim 36 wherein the reaction of a compound of Formula VI with an aldehyde of Formula VII to give a compound of Formula VIII is carried out in the presence of a suitable catalyst selected from the group comprising of sodium cyanide, thiazolium bromide and thiazolium chloride in a suitable solvent selected from the group comprising of methanol, ethanol, propanol and isopropanol.

46. The process according to claim 36 wherein the reaction of a compound of Formula VI with an aldehyde of Formula VII is carried out in the presence of a suitable base selected from the group comprising of, triethylamine and pyridine.

47. The process according to claim 46 is carried out in presence of triethylamine.

48. The process according to claim 36 wherein the reaction of a compound of Formula VIII with a compound of Formula IX to give a compound of Formula X is carried out in a suitable solvent selected from the group comprising of xylene and toluene.

49. The process according to claim 48 is carried out in toluene.

50. The process according to claim 36 wherein the reaction of a compound of Formula VIII with a compound of Formula IX is carried out in presence of an organic acid selected from the group comprising of pivalic acid and p-toluene sulfonic acid.

51. The process according to claim 36 wherein the conversion of a compound of Formula X to give a compound of Formula I is carried out (i) cleaving of ketal by acid catalysis and (ii) hydrolysis of the resulting ester.

52. The cleavage of ketal according to claim 51 is carried out in aqueous mineral acid.

53. The aqueous mineral acid according to claim 52 is aqueous hydrochloric acid.

54. The hydrolysis of ester according to claim 51 is carried out in the presence of a suitable base selected from the group comprising of lithium hydroxide, sodium hydroxide and potassium hydroxide.

55. A process for the preparation of compound of Formula I, 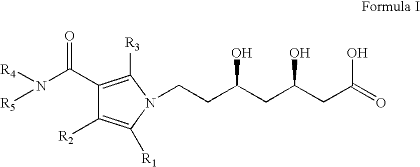
its pharmaceutically acceptable salts, pharmaceutically acceptable solvates, polymorphs or N-oxide wherein
R₁ is 4-fluorophenyl, 2,4-difluorophenyl, 3,4-difluorophenyl or cyclohexyl;
R₂ is phenyl, 4-fluorophenyl, 2,4-difluorophenyl, 4-methylphenyl or 4- trifluoromethylphenyl;
R₃ is isopropyl or cyclopropyl;
R₄ is hydrogen or methyl;
R₅ is phenyl, 2- fluorophenyl 3- fluorophenyl, 4-fluorophenyl, 2- pyridyl, 3- pyridyl, 4-pyridyl, 2-cyanophenyl, 4-cyanophenyl, 2,4-difluorophenyl, or 4-trifluoromethylphenyl, with the provisio that simultaneously R₁, R₂, R₃, R₄ and R₅ can not be respectively, 4-fluorophenyl, phenyl, isopropyl, hydrogen and phenyl comprising reacting a compound of Formula XI with a compound of Formula V to give a compound of Formula XII, 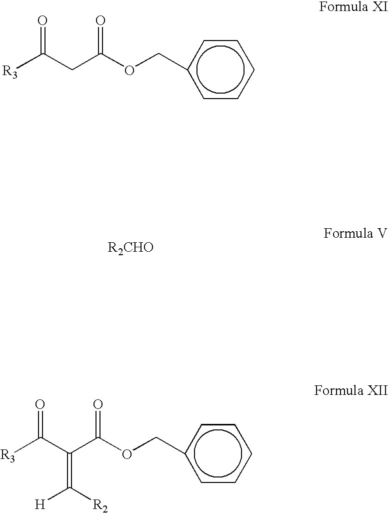
which on reaction with a compound of Formula VII gives a compound of Formula XIII, 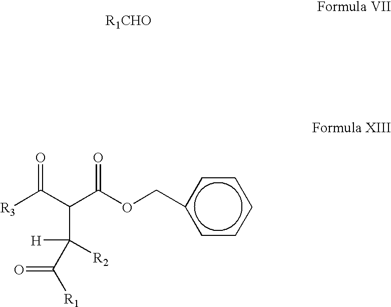
which on treatment with a compound of Formula IX yields a compound of Formula XIV, 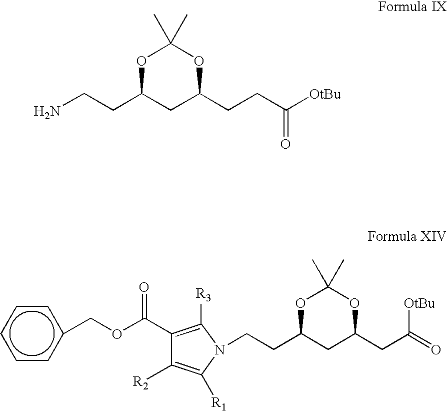
which on debenzylation gives a compound of Formula XV, 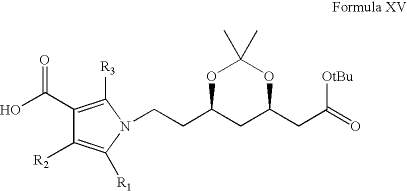
which on conversion to its acid chloride (Path a) or reacting with alkyl chloroformate (Path b) followed by reaction with a compound of Formula III gives a compound of Formula X, 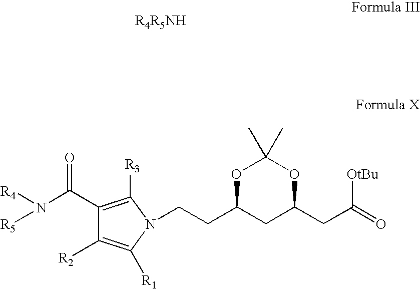
which on hydrolysis gives a compound of Formula I.

56. The process according to claim 55 wherein tthe reaction of a compound of Formula XI with an aldehyde of Formula V to give a compound of Formula XII is carried out in a suitable solvent selected from the group comprising of xylene, toluene, heptane, hexane and dichloromethane.

57. The process according to claim 55 wherein the reaction of a compound of Formula XI with a compound of Formula V is carried out in the presence of an organic base selected from the group comprising of triethylamine, pyridine, piperidine and β-alanine and an organic acid selected from group comprising of glacial acetic acid and benzoic acid.

58. The process according to claim 57 wherein the reaction is carried out in the presence of β-alanine and glacial acetic acid.

59. The process according to claim 55 wherein the reaction of a compound of Formula XII with an aldehyde of Formula VII to give a compound of Formula XIII is carried out in a suitable solvent selected from the group comprising of methanol, ethanol, propanol and isopropanol.

60. The process according to claim 55 wherein the reaction of a compound of Formula XII with an aldehyde of Formula VII is carried out in the presence of an organic base selected from the group comprising of, triethylamine, piperidine and pyridine.

61. The process according to claim 55 wherein the reaction of a compound of Formula XII with an aldehyde of Formula VII to give a compound of Formula XIII is carried out in the presence of a suitable catalyst selected from the group comprising of sodium cyanide, thiazolium bromide and thiazolium chloride.

62. The process according to claim 55 wherein the reaction of a compound of Formula XIII with an amine of Formula IX to give a compound of Formula XIV is carried out in the presence of an acid selected from the group comprising of pivalic acid and p-toluene sulfonic acid in a suitable solvent selected from the group comprising of hexane, heptane, toluene and tetrahydrofuran.

63. The process according to claim 55 wherein the debenzylation of a compound of Formula XIV to give a compound of Formula XV is carried out by hydrogenation in a suitable solvent selected from the group comprising of methanol, ethanol, propanol and dioxane.

64. The process according to claim 63 wherein the hydrogenation is carried out with palladium on carbon and hydrogen.

65. The process according to claim 55 wherein the conversion of a compound of Formula XV to its corresponding acid chloride is carried with a suitable chlorinating agent in a suitable solvent followed by reaction with a compound of Formula III to give a compound of Formula X in a suitable solvent and in the presence of an organic base.

66. The process according to claim 65 wherein the chlorinating agent is oxalyl chloride.

67. The process according to claim 65 wherein a suitable solvent is selected from the group comprising of benzene, toluene, xylene, chloroform, dichloromethane and tetrahydrofura

68. The process according to claim 65 wherein the organic base is selected from the group comprising of triethylamine and pyridine.

69. The process according to claim 54 wherein the reaction of compound of Formula XV with alkyl chloroformate is carried out in tetrahydrofuran.

70. The process according to claim 54 wherein the reaction of compound of Formula XV with alkyl chloroformate is carried out in the presence of triethylamine.

71. The process according to claim 54 wherein the alkyl chloroformate is selected from the group comprising of ethyl chloroformate, isopropyl chloroformate and isobutyryl chloroformate

72. A compound of Formula I. A process for the preparation of compound of Formula I, its pharmaceutically acceptable salts, pharmaceutically acceptable solvates, polymorphs or N-oxide wherein
R₁ is 4-fluorophenyl, 2,4-difluorophenyl, 3,4-difluorophenyl or cyclohexyl;
R₂ is phenyl, 4-fluorophenyl, 2,4-difluorophenyl, 4-methylphenyl or 4-trifluoromethylphenyl;
R₃ is isopropyl or cyclopropyl;
R₄ is hydrogen or methyl;
R₅ is phenyl, 2- fluorophenyl 3- fluorophenyl, 4-fluorophenyl, 2- pyridyl, 3- pyridyl, 4-pyridyl, 2-cyanophenyl, 4-cyanophenyl, 2,4-difluorophenyl, or 4-trifluoromethylphenyl, with the provisio that simultaneously R₁, R₂, R₃, R₄ and R₅ can not be respectively, 4-fluorophenyl, phenyl, isopropyl, hydrogen and phenyl.
